(19) United States
(12) Patent Application Publication
(10) Pub. No.: US 2015/0142019 A1
(43) Pub. Date: May 21, 2015

(54) MEDICAL SUTURING INSTRUMENT AND RELATED METHODS THEREFOR

(71) Applicant: COVIDIEN LP, MANSFIELD, MA (US)

(72) Inventor: Yosuke Sakai, Shizuoka (JP)

(21) Appl. No.: 14/406,464

(22) PCT Filed: Jun. 14, 2013
(86) PCT No.: PCT/US13/45798

§ 371 Date: Dec. 8, 2014

(30) Foreign Application Priority Data
Jun. 18, 2012 (JP) 2012-137185

(51) Int. Cl. A61B 17/062

(52) U.S. Cl. CPC A61B 17/062; A61B 2017/00526
USPC 606/146; 29/428

(57) ABSTRACT

A medical suturing instrument comprises: a loop introduction needle; a suture thread introduction needle into which suture thread is inserted; a housing for holding the loop introduction needle and the suture thread introduction needle substantially parallel a prescribed distance apart; a loop forming part which has a loop formed at the tip end thereof and is movably housed inside the loop introduction needle; a loop feed/return mechanism provided inside the housing for moving the loop forming part in the axial direction; a suture thread feed mechanism provided inside the housing for feeding out the suture thread on the tip end side; and a thread housing part provided in the housing for accommodating the suture thread which is supplied to the suture thread feed mechanism.

### BACKGROUND

1. Technical Field

The present invention relates to a medical suturing instrument which is used when suture thread for holding the wall of an internal organ such as the stomach or bladder from outside the body on the body surface-side, such as the abdominal wall, is introduced into the internal organ, and when the suture thread is withdrawn from inside the internal organ.

2. Related Art

People with a reduced function for ingesting food orally under their own power due to advanced age or illness (referred to below as “patients”) are administered with enteral feeding in which liquid food and nutrients etc. are supplied using a gastrostomy catheter. When percutaneous endoscopic gastrostomy (PEG) is used, for example, a through-hole (e.g., a fistula such as a gastric fistula) which runs through the patient's abdominal wall and the wall of an internal organ (stomach wall) is established, the gastrostomy catheter is fitted into the through-hole, and the patient is supplied with liquid food etc. through the gastrostomy catheter.

When the through-hole is established, the wall of an internal organ which readily moves and the abdominal wall are normally sutured and fixed percutaneously using suture thread in order to simplify formation of the through-hole. Various kinds of medical suturing instruments have been proposed for use in suturing and fixing the internal organ wall and the abdominal wall.

An instrument of this kind which is disclosed is a medical instrument “which houses, inside a case main body, a feed-out mechanism for successively feeding out towards the tip end a suture thread inserted into a suture thread insertion puncture needle 20 from the base end thereof; and a projection mechanism for causing an annular member of a stylet housed inside a suture thread gripping puncture needle 30 to project from the tip end of the suture thread gripping puncture needle 30,” for example, as described with reference to FIGS. 5 and 6 in Japanese Patent Application Nos. JP2009-213763A and JP2009-213764A. This medical instrument is designed to make it possible for only one practitioner or for a practitioner and an assistant to efficiently and safely form a ligature inside the body.

Operation of the medical instruments, such as those disclosed in the above-referenced Japanese patent applications, requires multiple steps in which: punctures are made in the stomach from the abdominal wall using the suture thread insertion puncture needle and the suture thread gripping puncture needle, after which a stylet is pushed in in order to form the annular shape of the annular member; an operating roller is turned in order to insert the suture thread into the stomach; and a release button is pressed in order to grip the suture thread with the annular member, all of which make the operating procedure more extensive.

If the internal organ wall and the abdominal wall are to be fixed at multiple sites with medical instruments, such as those disclosed in the above-referenced Japanese patent applications, the practitioner has to insert the suture thread into the medical instrument on each occasion, which increases work for the practitioner

### SUMMARY

One or more aspects of the present invention can be directed to a medical suturing instrument that reduces the practitioner's work burden when the suture thread is inserted into the medical instrument relative to those described, for example, in Japanese Patent Application Nos. JP2009-213763A and JP2009-213764A. For example, according to the embodiments described herein, operation of the suturing instrument automatically dispenses suture thread stored in a thread housing part coupled to a main housing of the suturing instrument. Such an approach frees the practitioner or an assistant from the labor and attention required to feed suture thread into the suturing instrument when in the instrument is in use. The preceding makes a stitching operation on a body and associated internal organs more failsafe and faster to complete.

According to one or more aspects, a medical suturing instrument includes a loop introduction needle, a suture thread introduction needle configured to receive suture thread inserted therewithin, a housing configured to hold the loop introduction needle and the suture thread introduction needle substantially parallel a prescribed distance apart, a loop forming part having a loop formed at the tip end thereof, the loop forming part movably housed inside the loop introduction needle, a loop feed/return mechanism located inside the housing and configured to move the loop forming part in an axial direction, a suture thread feed mechanism located inside the housing and configured to feed out the suture thread on a tip end side; and a thread housing part coupled to the housing for accommodating the suture thread which is supplied to the suture thread feed mechanism.

In the medical suturing instrument according to one or more aspects, an operation part is included, where the operation part is configured to receive input operations. In some embodiments, the operation part is configured to move in a forward direction on receipt of an input operation to move the loop feed/return mechanism into feeding the loop out from the loop introduction needle, and to move the suture thread feed mechanism into feeding the suture thread out from the suture thread introduction needle after at least part of the loop has projected from a tip end of the loop introduction needle, the suture thread feed mechanism is configured to operate to enable the suture thread to freely advance and retract after the loop is fed out from the loop introduction needle and the suture thread is fed out from the suture thread introduction needle, and the loop feed/return mechanism is configured to operate to return the loop inside the loop introduction needle after the loop is fed out from the loop introduction needle and the suture thread is fed out from the suture thread introduction needle.

According to some further embodiments, the medical suturing instrument includes a bobbin located inside the thread housing and configured to have the suture thread wound thereon and to rotate in a winding direction of the suture thread, and a pin located inside the thread housing part and extending in a direction which intersects a winding direction of the suture thread when housed in a wound state inside the thread housing part.

According to some other embodiments, the thread housing part includes a peripheral wall which has a substantially circular planar shape, a thread dispensing opening configured to dispense the suture thread inside the thread housing part and located in the peripheral wall of the thread housing part, and a thread insertion opening located in the housing and configured to receive suture thread inserted into the housing. According to these embodiments, the thread dispensing opening and the thread insertion opening are located in such a way that when viewed as a plane, an imaginary line joining the two substantially coincides with the tangential direction of the peripheral wall at the thread dispensing opening.

According to a further embodiment, the medical suturing instrument includes a bobbin located inside the thread housing, the bobbin configured to have the suture thread wound thereon and to rotate in a winding direction of the suture thread.

According to one or more further aspects, a method for thread-feeding and thread-storage in a medical suturing instrument is provided where the medical suturing instrument includes a loop introduction needle, a suture thread introduction needle, a housing configured to hold the loop introduction needle and the suture thread introduction needle, a loop forming part having a loop at a tip end thereof, a loop feed/return mechanism inside the housing, a suture thread feed mechanism inside the housing and configured to feed out the suture thread on a tip end side and a thread housing part located in the housing. According to some embodiments, the method includes acts of: coupling the loop introduction needle and the suture thread introduction needle to the housing substantially parallel to one another a prescribed distance apart; movably housing the loop forming part within the loop introduction needle; housing the suture thread in the thread housing part; operating the medical suturing instrument to move the loop forming part in an axial direction with the loop feed/return mechanism; and operating the medical suturing instrument to withdraw suture thread from the thread housing part and feed the suture thread into the suture thread introduction needle using the suture thread feed mechanism.

In the method according to one or more aspects, the medical suturing instrument further comprises a bobbin located within the thread housing part and an operating part, and the method further includes: rotating the bobbin in a winding direction of the suture thread to wind the suture thread on the bobbin; moving the operating part in a forward direction on receipt of an input operation; moving the loop feed/return mechanism into feeding the loop out from the loop introduction needle; moving the suture thread feed mechanism into feeding the suture thread out from the suture thread introduction needle after at least part of the loop has projected from a tip end of the loop introduction needle; operating the suture thread feed mechanism to enable the suture thread to freely advance and retract after the loop is fed out from the loop introduction needle and the suture thread is fed out from the suture thread introduction needle; and operating the loop feed/return mechanism to return the loop inside the loop introduction needle after the loop is fed out from the loop introduction needle and the suture thread is fed out from the suture thread introduction needle.

According to another embodiment of the method according to one or more aspects, the method further includes locating a pin inside the thread housing part extending in a direction which intersects a winding direction of the suture thread when housed in a wound state inside the thread housing part. According to still another embodiment, the method includes: including a thread insertion opening in the housing; including a peripheral wall having a substantially circular planar shape and a thread dispensing opening located therein within the thread housing part; locating the thread dispensing opening and the thread insertion opening relative to one another such that, when viewed as a plane, an imaginary line joining the two substantially coincides with the tangential direction of the peripheral wall at the thread dispensing opening; and dispensing the suture thread via the thread dispensing opening and inserting the suture thread into the housing via the thread insertion opening.

The medical suturing instrument according to the present invention comprises: a loop introduction needle; a suture thread introduction needle into which suture thread is inserted; a housing for holding the loop introduction needle and the suture thread introduction needle substantially parallel a prescribed distance apart; a loop forming part which has a loop formed at the tip end thereof and is movably housed inside the loop introduction needle; a loop feed/return mechanism provided inside the housing for moving the loop forming part in the axial direction; a suture thread feed mechanism provided inside the housing for feeding out the suture thread on the tip end side; a thread housing part provided in the housing for accommodating the suture thread on the upstream side of the suture thread feed mechanism; and an operation part which causes operation of the loop feed/return mechanism and the suture thread feed mechanism; when the operation part receives an input operation, the loop feed/return mechanism operates in such a way that the loop projects from the tip end of the loop introduction needle, and after at least part of the loop has projected from the tip end of the loop introduction needle, the suture thread feed mechanism also operates in such a way that the suture thread projects from the tip end of the suture thread introduction needle; after this, the suture thread feed mechanism operates in such a way as to enable the suture thread to freely advance and retract; and after this, the loop feed/return mechanism operates in such a way that the loop returns inside the loop introduction needle.

The medical suturing instrument according to the present invention enables the suture thread to be introduced into an internal organ and to be withdrawn therefrom when the operation part receives an input operation, and therefore the work burden of the practitioner can be considerably reduced. Furthermore, the medical suturing instrument according to the present invention is provided with a thread housing part for accommodating the suture thread, so when there are repeated operations to introduce the suture thread into an internal organ, it is no longer necessary to insert the suture thread into the medical suturing instrument on each occasion, to reduce the practitioner's work burden.

According to one or more still further aspects, a method of making a medical suturing instrument, includes acts of: providing each of a loop introduction needle and a suture thread introduction needle, the suture thread needle configured to suture thread inserted therewithin; housing the loop introduction needle and the suture thread introduction needle, at least partly, in a housing such that loop introduction needle and the suture thread introduction needle are held substantially parallel to one another a prescribed distance apart; locating a loop forming part inside the loop introduction needle, the loop forming part having a loop formed at a tip end thereof, the loop forming part movably housed inside the loop introduction needle; locating each of a loop feed/return mechanism and a suture thread feed mechanism inside the housing, the loop fed return mechanism configured to move the loop forming part in an axial direction, the suture thread feed mechanism configured to feed out the suture thread on a tip end side; and coupling a thread housing part to the housing, the thread housing part configured to accommodate a suture thread which is supplied to the suture thread feed mechanism.

### BRIEF DESCRIPTION OF THE DRAWINGS

FIG. 1 is an external oblique view seen from above showing one example of the structure of the medical suturing instrument according to one or more embodiments of the invention;

FIG. 2 is an external oblique view seen from below of the medical suturing instrument according to one or more embodiments of the invention;

FIG. 3 is a side view of the medical suturing instrument according to one or more embodiments of the invention;

FIG. 4 is a bottom view of the medical suturing instrument according to one or more embodiments of the invention;

FIG. 5 is a back view of the medical suturing instrument according to one or more embodiments of the invention;

FIG. 6 is a schematic internal structural diagram showing one example of the internal structure of the medical suturing instrument according to one or more embodiments of the invention;

FIG. 7 is a schematic view in cross section along A-A in FIG. 4;

FIGS. 8A-8C illustrate the structure of the bobbin according to one or more embodiments of the invention;

FIGS. 9A-9C illustrate an example of the attachment position of the thread housing part according to one or more embodiments of the invention;

FIG. 10 is a schematic view in cross section showing, in simplified form, the procedure for operating the medical suturing instrument according to one or more embodiments of the invention;

FIGS. 11A-11C are schematic views in cross section showing, in simplified form, the procedure for operating the medical suturing instrument according to one or more embodiments of the invention;

FIGS. 12A-12C are schematic views in cross section showing, in simplified form, the procedure for operating the medical suturing instrument according to one or more embodiments of the invention;

FIG. 13A-13C are schematic views in cross section showing, in simplified form, the procedure for operating the medical suturing instrument according to one or more embodiments of the invention;

FIGS. 14A-14C are explanatory diagrams to illustrate the action of the medical suturing instrument according to one or more embodiments of the invention;

FIG. 15 is a bottom view of the medical suturing instrument according to one or more embodiments of the invention; and

FIG. 16 is a view in cross section along B-B in FIG. 15.

### DETAILED DESCRIPTION

Non-limiting embodiments of the present invention will be exemplarily described below with reference to the accompanying drawings.

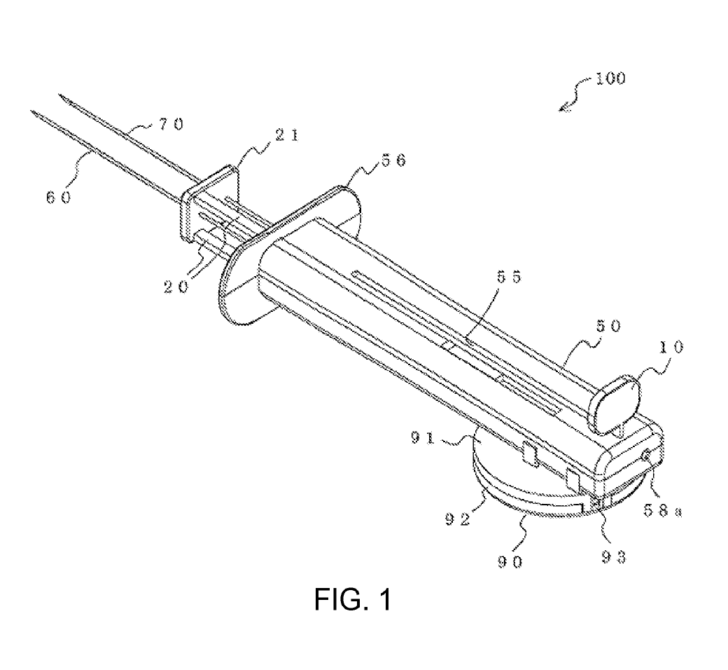

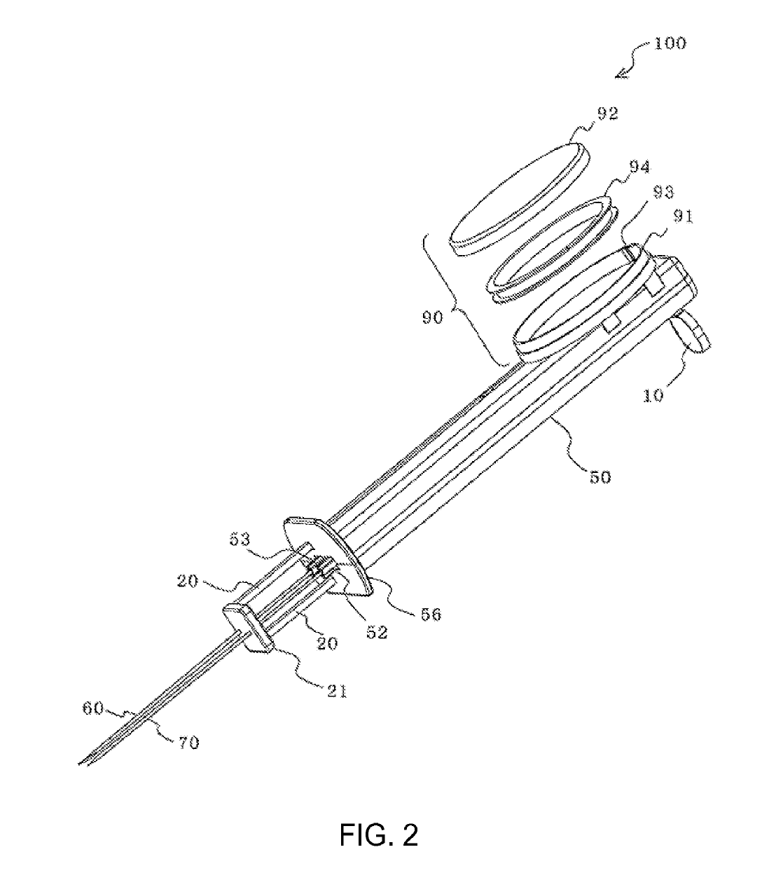

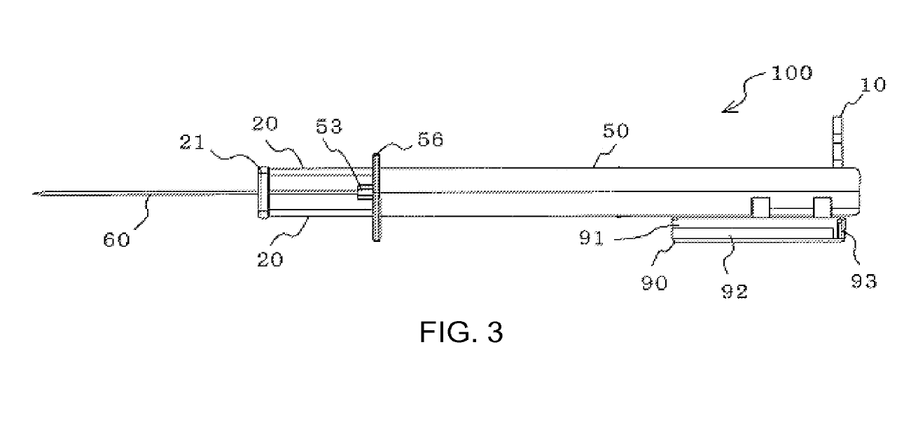

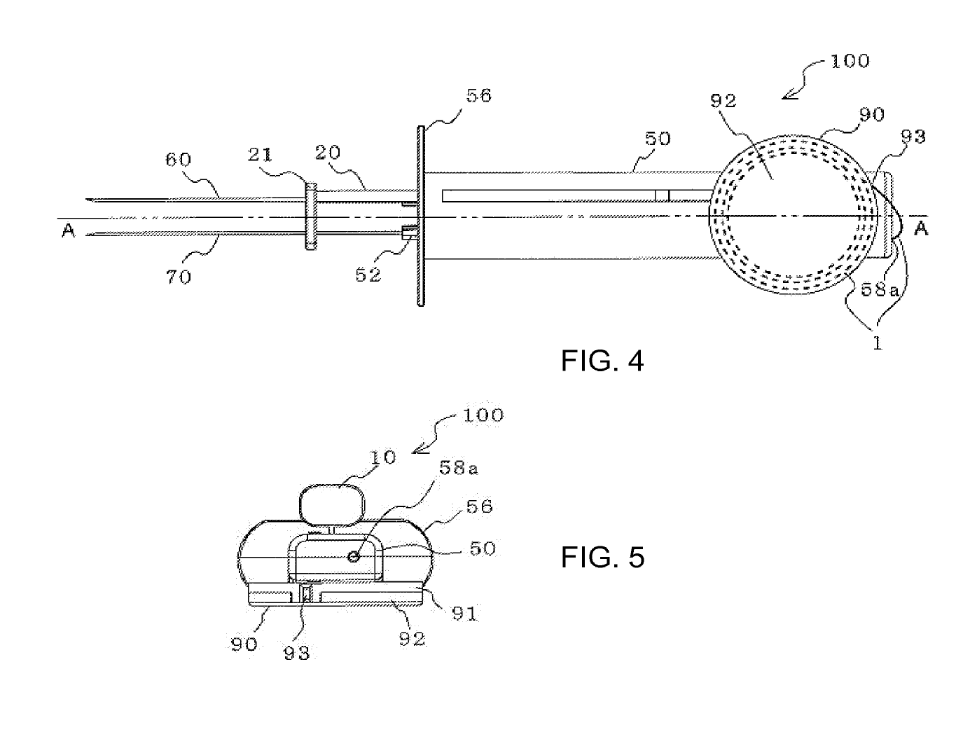

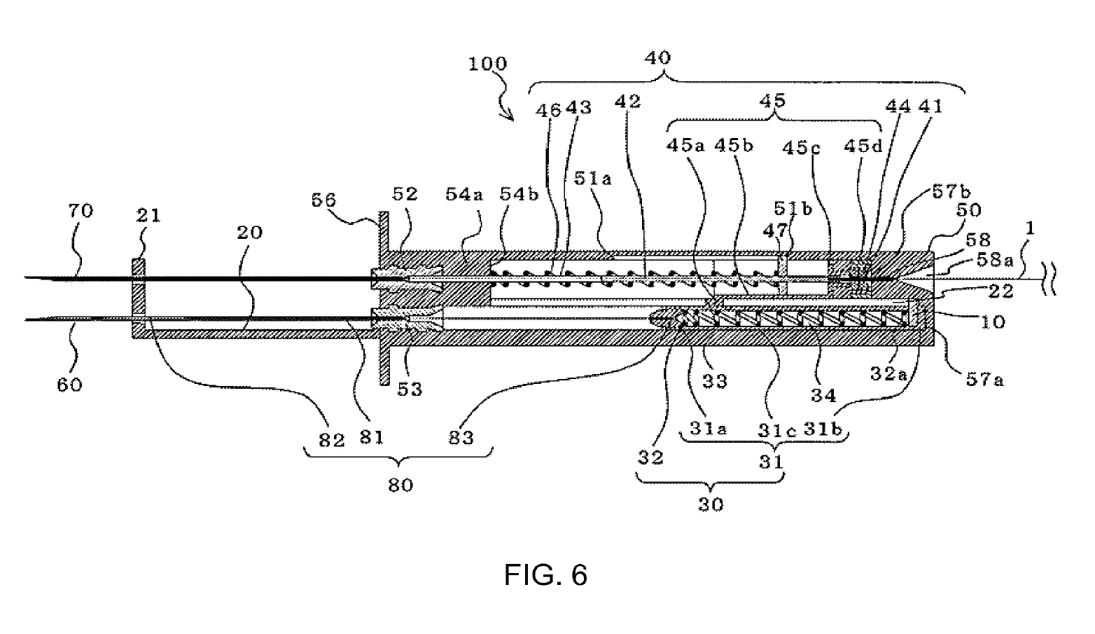

FIG. 1 is an external oblique view seen from above showing one example of the structure of the medical suturing instrument according to an embodiment of the present invention (referred to below simply as the “suturing instrument 100”). FIG. 2 is an external oblique view seen from below of the medical suturing instrument 100, showing a partially exploded state. FIG. 3 is a side view of the medical suturing instrument 100. FIG. 4 is a bottom view of the medical suturing instrument 100. FIG. 5 is a back view of the medical suturing instrument 100. FIG. 6 is a schematic internal structural diagram showing one example of the internal structure of the medical suturing instrument 100. The structure and operation of the suturing instrument 100 will be described with reference to FIGS. 1 to 6. The suturing instrument 100 is used in order to facilitate formation of a fistula for insertion of a fistula catheter when suture thread for lifting the wall of an internal organ such as the stomach or bladder from outside the body towards the abdominal wall and holding it in place is introduced into the internal organ. It should be noted that the size relationships of the structural components in the figures below may differ from the actual size relationships, including in FIG. 1.

The suturing instrument 100 externally comprises: a housing 50; an operation part 10 which is operated by the practitioner; a loop introduction needle 60 which is provided at the tip end of the housing 50; a suture thread introduction needle 70 which is provided at the tip end of the housing 50 a prescribed distance apart from the loop introduction needle 60 and substantially parallel thereto; and a thread housing part 90. The suturing instrument 100 also comprises, inside the housing 50, a loop feed/return mechanism 30 which operates in conjunction with the movement of the operation part 10; and a suture thread feed mechanism 40 which operates in conjunction with the movement of the operation part 10. The suturing instrument 100 further comprises a loop forming part 80 which is movably inserted inside the loop introduction needle 60.

It should be noted that in the following description, the tip end side denotes the side which is inserted into the patient (patient side), while the base end side denotes the side which is operated by the practitioner (operation side). Furthermore, in the description which follows, the tip end side may also be referred to as forwards or the front, and the base end side may also be referred to as backwards or the rear.

Furthermore, FIGS. 4 and 6 also schematically depict suture thread 1 which is fed out from the tip end (point) of the suture thread introduction needle 70 by means of the suture thread feed mechanism 40.

The suture thread 1 does not form part of the suturing instrument 100, but it will be described briefly as it serves as an organopexy tool. The suture thread 1 should be made of a material which is sufficiently flexible that it can bend along bodily tissues when inserted inside the body, and which also has sufficient tensile strength that it can lift internal organs (nylon yarn, for example). Furthermore, the suture thread 1 is cut when fitted to or removed from the patient. For this reason, the suture thread 1 is preferably made of a material and has a diameter size that can be cut by an instrument found in a medical setting, such as scissors. In addition, the suture thread 1 has the length required for multiple fixing operations. As will be described later, the practitioner punctures the patient with the suturing instrument 100 while the suture thread 1 is inserted up to the tip end of the suture thread introduction needle 70.

The housing 50 has a hollow, substantially cuboid shape which is rectangular in the front-to-rear direction. The housing 50 accommodates the loop feed/return mechanism 30 and the suture thread feed mechanism 40. The loop feed/return mechanism 30 and the suture thread feed mechanism 40 are accommodated substantially in parallel inside the housing 50, and they can move backwards and forwards inside the housing 50. The housing 50 forms the unit of the suturing instrument 100 as a whole and constitutes the part which is actually held by the practitioner during a procedure. It should be noted that the housing 50 does not have to be substantially cuboid in shape, and there is no particular limitation as to the external shape thereof. Furthermore, the housing 50 may simply consist of a skeleton. That is, the housing 50 should have a structure which enables the other components (to be described later) to function.

Furthermore, the loop introduction needle 60 and the suture thread introduction needle 70 are fitted a prescribed distance apart and substantially parallel to each other at the tip end of the housing 50. For example, as shown in FIG. 3, support parts (support part 52, support part 53) may be provided at the tip end of the housing 50, and the loop introduction needle 60 and the suture thread introduction needle 70 may be inserted into the support parts so that the loop introduction needle 60 and the suture thread introduction needle 70 are detachably supported. The support parts should, for example, comprise a material such as synthetic resin which is moulded into a substantially cuboid shape, a substantially cylindrical shape, or a plurality of these shapes combined (a plurality of substantially cuboid shapes combined, a plurality of substantially cylindrical shapes combined, or a combination of substantially cuboid shapes and substantially cylindrical shapes). Furthermore, multiple protrusions may be provided on the outer periphery of the support parts, as shown in the figures. It should be noted that the support parts may equally be designed in such a way as not to project from the tip end side of the housing 50.

Moreover, the base ends of the loop introduction needle 60 and the suture thread introduction needle 70 may already be supported by support members which are detachably fitted to the tip end of the housing 50. Furthermore, the loop introduction needle 60 and the suture thread introduction needle 70 may already be fixed to the housing 50. In addition, the loop introduction needle 60 and the suture thread introduction needle 70 do not have to be fitted to the very tip end of the housing 50, provided that they are fitted towards the tip end of the housing 50. That is, the base ends of the loop introduction needle 60 and the suture thread introduction needle 70 should extend into the housing 50.

The tip end side of the suture thread feed mechanism 40 in the housing 50 has a predetermined thickness such that a third hollow member 43 which forms part of the suture thread feed mechanism 40 is supported as it passes therethrough. The section having a predetermined thickness on the tip end side of the suture thread feed mechanism 40 in the housing 50 is referred to as the “thick-walled part 54a”. Furthermore, the inner wall surface on the tip end side of the suture thread feed mechanism 40 in the housing 50, that is, the surface of the thick-walled part 54a on the suture thread feed mechanism 40 side, is simply referred to as the “inner wall surface 54b”. In other words, in the construction of the housing 50 shown in FIG. 3, the inner wall surface 54b is formed only on the suture thread feed mechanism 40 side. However, the construction of the housing 50 is not limited to what is shown in FIG. 3, and the thick-walled part 54a may be provided on the loop feed/return mechanism 30 side.

The tip end of a second elastic member 46 which forms part of the suture thread feed mechanism 40 abuts the inner wall surface 54b. Furthermore, a first abutment part 33 which forms part of a loop feed/return part 31 (to be described later) also abuts the inner wall surface 54b. When the first abutment part 33 which forms part of the loop feed/return part 31 abuts the inner wall surface 54b, the loop feed/return part 31 does not move any further forwards.

A through-hole 58 for supporting a first hollow member 41 which forms part of the suture thread feed mechanism 40 is formed in the base end-side wall of the housing 50 on the suture thread feed mechanism 40 side. Part of the base end side of the first hollow member 41 is fixed to part of the inner peripheral surface of the through-hole 58, and the suture thread 1 which has been inserted from an end opening (thread insertion opening 58a) on the base end side of the through-hole 58 is inserted into the cavity of the first hollow member 41. In the example shown in FIG. 6, the inner diameter of a region of predetermined length on the base end side of the through-hole 58 is formed as a taper which expands towards the end opening (referred to as the “thread insertion opening 58a”) on the base end side of the through-hole 58, and therefore it is a simple matter for the practitioner to insert the suture thread 1 into the thread insertion opening 58a.

However, the support structure for the first hollow member 41 is not limited to the embodiment illustrated in FIG. 6, and the first hollow member 41 may be supported without the tapered portion being formed in the through-hole 58, or the base end of the first hollow member 41 may be supported in such a way as to project outside the housing 50. Furthermore, a support member similar to the support parts (support part 52, support part 53) may be attached to the base end side of the housing 50 on the suture thread feed mechanism 40 side in order to support the first hollow member 41. In this case, the first hollow member 41 may be attached to or detached from the housing 50 after attachment to the support member.

Furthermore, a stopper 51a and a stopper 51b are formed on part of the inner wall surface of the housing 50. The stopper 51a and the stopper 51b may be formed to cause part of the inner wall surface of the housing 50 to project into the housing 50, or may be formed by providing a step on part of the inner wall surface of the housing 50. The stopper 51a and the stopper 51b have the function of regulating the forwards and backwards movement of the movement regulating member 47 which is fixed to the second hollow member 42. However, the stopper 51a is not required if the forwards movement of the second hollow member 42 is regulated by the tip end of the second hollow member 42 abutting the support part 52 or inner wall surface 54b, or abutting part of the inner wall of a third hollow member 43, or by the movement regulating member 47 abutting the base end of the third hollow member 43. It should be noted that if the tip end of the second hollow member 42 is made to abut the inner wall surface 54b, the third hollow member 43 is not provided.

Likewise, the stopper 51b is not required if the backwards movement of the second hollow member 42 is regulated by the base end of the second hollow member 42 abutting the outer wall of a first hollow member 41, or the tip end of the second hollow member 42 abutting the base end (inner side) of the third hollow member 43. Furthermore, if the tip end of the second hollow member 42 comes into abutment with the base end (inner side) of the third hollow member 43, the tip end of the second hollow member 42 projects radially outwards and the base end of the third hollow member 43 projects radially inwards, so the backwards movement of the second hollow member 42 may be regulated by the engagement of the two. It should be noted that the stopper 51a corresponds to the first stopper according to the present invention, and the stopper 51b corresponds to the second stopper according to the present invention.

Furthermore, a guide hole 55 which allows the operation part 10 to move forwards and backwards is formed in the lengthwise direction of the housing 50 in the side surface thereof. The guide hole 55 runs through the side surface of the housing 50 and is formed with a prescribed length. Moreover, there is no particular limitation as to the material from which the housing 50 is made, and a polyolefin such as polypropylene or polyethylene, or a synthetic resin such as polyvinyl chloride or polycarbonate may be employed. Moreover, a flange part 56 which projects radially outwards in the form of a rim is provided on the outer wall at the tip end of the housing 50. The flange part 56 serves to assist in the puncture operation.

A support rod 20 allowing forwards and backwards movement of a flat plate part 21 which is penetrated by the loop introduction needle 60 and the suture thread introduction needle 70 is provided on the housing 50. The flat plate part 21 can restrict changes in the distance between the loop introduction needle 60 and the suture thread introduction needle 70 by moving towards the tip end when the loop introduction needle 60 and the suture thread introduction needle 70 are made to pierce the patient's body. By providing this kind of flat plate part 21, it is possible to prevent flexing of the loop introduction needle 60 and the suture thread introduction needle 70 as well as any large changes in the distance therebetween. Furthermore, through the support rod 20, the flat plate part 21 can move forwards and backwards with respect to the loop introduction needle 60 and the suture thread introduction needle 70, so the flat plate part 21 does not present an obstruction to the piercing operation.

There is no particular limitation as to the planar shape of the flat plate part 21, but it may be rectangular, circular or polygonal. Furthermore, the tip end surface of the flat plate part 21 (the surface on the patient side) should be flat so as not to irritate the patient's skin. When this kind of flat plate part 21 is provided, the tip end of the support rod 20 extending parallel to the loop introduction needle 60 and the suture thread introduction needle 70 should be fixed to the flat plate part 21. Providing the support rod 20 makes it possible to reduce the force transmitted from the flat plate part 21 to the loop introduction needle 60 and the suture thread introduction needle 70, while also improving the stability of movement of the flat plate part 21.

The operation part 10 is operated by the practitioner when an organopexy tool is introduced into an internal organ using the suturing instrument 100. Furthermore, the operation part 10 serves to transmit the force applied by the practitioner to the loop feed/return mechanism 30 and the suture thread feed mechanism 40. The operation part 10 is disposed between the base end part 32a of a first elastic member 32 which forms part of the loop feed/return mechanism 30, and the base end part 31b of the loop feed/return part 31. The operation part 10 is provided partly inside the housing 50 or partly in the guide hole 55 of the housing 50 projecting from part of the side surface of the housing 50. Furthermore, the operation part 10 is able to move forwards and backwards along the guide hole 55. It should be noted that FIGS. 1 to 3 show an exemplary situation in which part of the operation part 10 is formed projecting from the guide hole 55.

Moreover, there is no particular limitation as to the material from which the operation part 10 is made, and it may be made from the same synthetic resin as the housing 50, or from a different material to that of the housing 50. Furthermore, there is no particular limitation as to the shape of the operation part 10, provided that it is a shape which can receive operations from the practitioner and allows practitioner operations to be transmitted to the loop feed/return mechanism 30 and the suture thread feed mechanism 40. Furthermore, a guide for moving the operation part 10 forwards and backwards in a straight line may be formed inside the housing 50 (the groove 22 shown in FIG. 3, for example).

FIGS. 1 to 3 show an example of the operation part 10 having a shape which projects outwards from the side surface of the housing 50 in a direction orthogonal to the direction of advance. By adopting such a shape, it is easy for the practitioner to catch the operation part 10 with the fingers, and the handling is improved. However, the shape of the operation part 10 is not restricted to the shape shown in FIGS. 1 to 3, and it is possible to extend the base end of the operation part 10 to the rear and to provide a rod which projects from the base end of the housing 50 so that the base end of the rod can be retractably inserted into the housing 50 to serve as the operation part 10. A button or the like may be provided at the base end of the rod in order to improve the handling. Furthermore, if the operation part 10 is provided inside the housing 50, a finger may be inserted through the guide hole 55 in order to operate the operation part 10.

The loop feed/return mechanism 30 operates in conjunction with the movement of the operation part 10 to cause the loop forming part 80, which is movably inserted into the loop introduction needle 60, to be fed out towards the tip end of the loop introduction needle 60 or to be returned inside the housing 50. The loop feed/return mechanism 30 comprises the loop feed/return part 31 and the first elastic member 32. It should be noted that the first elastic member 32 corresponds to the elastic member according to the present invention.

The loop feed/return part 31 comprises a tip end part 31a, the base end part 31b, a side wall part 31c, a first abutment part 33 and an accommodation part 34, and it can move forwards and backwards inside the housing 50 in response to the movement of the operation part 10. Moreover, the loop feed/return part 31 may, for example, comprise a hollow columnar member (cylindrical member) or a prismatic member (rectangular cylinder) or the like which can move forwards and backwards and in which the accommodation part 34 is formed such that the first elastic member 32 can be disposed therein. Furthermore, a cut (or incision) may be made in the side wall part 31c of the loop feed/return part 31 in order to allow communication between the accommodation part 34 and the outside. FIG. 3 shows an example of the loop feed/return part 31 with the suture thread feed mechanism 40 side open.

The tip end part 31a forms the tip end portion of the loop feed/return part 31, and a base end fixing part 83 of the loop forming part 80 is fixed thereto. Part of the tip end part 31a is inserted into the support part 53 when the loop feed/return part 31 has been moved forwards. The base end part 31b forms the base end portion of the loop feed/return part 31 and the operation part 10 is disposed on the accommodation part 34 side thereof. The base end part 31b regulates the backwards movement of the loop feed/return part 31. That is, when the base end part 31b runs into the base end inner wall surface of the housing 50 (referred to below simply as the “inner wall surface 57a”), the loop feed/return part 31 cannot move any further backwards. It should be noted that the positions of the base end inner wall surfaces may differ on the loop feed/return mechanism 30 side and the suture thread feed mechanism 40 side, so the base end inner wall surface of the housing 50 on the suture thread feed mechanism 40 side will be referred to as the “inner wall surface 57b”.

The side wall part 31c connects the tip end part 31a and the base end part 31b, and slidably abuts the inner wall surface inside the housing 50. However, the side wall part 31c does not have to cover the whole of the accommodation part 34, and a cut (or incision) may be formed therein in order to allow communication between the accommodation part 34 and the outside.

The first abutment part 33 has a base end surface in abutment with the tip end surface of a second abutment part 45a formed on a suture thread feed part 45 which forms part of the suture thread feed mechanism 40, and causes the suture thread feed part 45 to move backwards. Furthermore, as described above, the first abutment part 33 regulates the forwards movement of the loop feed/return part 31 by abutment with the inner wall surface 54b. The first abutment part 33 may be formed in the shape of a protrusion which projects from part of the side wall part 31c lying on the suture thread feed mechanism 40 side of the loop feed/return part 31, for example. Moreover, the shape of the first abutment part 33 is not limited to that of a protrusion, and it is sufficient for it to be able to transmit a backwards force to the suture thread feed part 45. Furthermore, the timing at which the first abutment part 33 and second abutment part 45a are made to abut will be described in detail at a later stage.

The accommodation part 34 is a space enclosed by the tip end part 31a, base end part 31b and side wall part 31c of the loop feed/return part 31, and the first elastic member 32 is extendibly accommodated therein.

The first elastic member 32 consists of an elastic member which is extendible in the front-to-rear direction (for instance, a spring (coil spring, air spring etc.) or a rubber element). The first elastic member 32 is accommodated in the accommodation part 34 in a compressed state in which a force greater than the friction force of the loop 82 accommodated in the loop introduction needle 60 is applied thereto. That is, when the first elastic member 32 is accommodated in the accommodation part 34, it does not deform unless a force greater than the friction force of the loop 82 is applied thereto. This means that while the first elastic member 32 is accommodated in the accommodation part 34, in other words while the operation part 10 is positioned between the first elastic member 32 and the base end part 31b, the loop feed/return part 31 is able to move forwards and backwards.

Moreover, a guide (a groove into which the loop feed/return part 31 fits, for example) for moving the loop feed/return part 31 forwards and backwards in a straight line may be formed inside the housing 50.

Moreover, forwards movement of the loop feed/return part 31 may be regulated by the tip end part 31a abutting the support part 53. Furthermore, if a thick-walled part 54a is also provided on the loop feed/return mechanism 30 side, forwards movement of the loop feed/return part 31 may be regulated by the tip end part 31a of the loop feed/return part 31 abutting the inner wall surface 54b rather than the support part 53.

The suture thread feed mechanism 40 operates in conjunction with movement of the operation part 10 in order to feed out, from the tip end (blade edge) of the suture thread introduction needle 70, suture thread (suture thread 1 shown in FIGS. 1 to 3) which is movably inserted into the suture thread introduction needle 70. The suture thread feed mechanism 40 comprises the first hollow member 41, the second hollow member 42, the third hollow member 43, a suture thread clamping member 44, the suture thread feed part 45, a second elastic member 46, and the movement regulating member 47.

The first hollow member 41 has a hollow shape and is fixed in the through-hole 58 provided on the base end side of the housing 50. The second hollow member 42 has a hollow shape and can move forwards and backwards inside the housing 50 in response to movement of the operation part 10. The third hollow member 43 has a hollow shape and is fixed to the tip end side of the housing 50. The suture thread 1 is inserted from outside the housing 50 into the first hollow member 41, second hollow member 42 and third hollow member 43. The third hollow member 43 in particular functions as a means for preventing flexing of the suture thread 1. However, the third hollow member 43 may be omitted so that the suture thread 1 is exposed inside the housing 50.

The second hollow member 42 has a larger inner diameter than the outer diameter of the first hollow member 41, and a smaller outer diameter than the inner diameter of the third hollow member 43. The second hollow member 42 can then move forwards and backwards when the tip end thereof is inserted into the hollow part of the third hollow member 43. Furthermore, the second hollow member 42 is designed to have the first hollow member 41 inserted into the hollow part at the base end side thereof, depending on the position of the second hollow member 42. In addition, the axial lengths of the first hollow member 41, second hollow member 42 and third hollow member 43 are determined in consideration of the size of the housing 50 and the length of the suture thread 1 which is to be projected from the tip end of the suture thread introduction needle 70, and there is no particular limitation to these lengths.

The movement regulating member 47 is fixed to the outer periphery on the base end side of the second hollow member 42. The movement regulating member 47 regulates the forwards and backwards movement of the second hollow member 42. That is, the second hollow member 42 moves forwards and backwards between abutment of the movement regulating member 47 with the stopper 51a and the stopper 51b formed on the inner wall surface of the housing 50. According to this embodiment, the forwards movement of the second hollow member 42 is regulated by the movement regulating member 47 abutting the stopper 51a. A description will be given below in which the backwards movement of the second hollow member 42 is regulated by the movement regulating member 47 abutting the stopper 51b in accordance with one embodiment.

Moreover, the forwards movement of the second hollow member 42 may be regulated by the tip end of the second hollow member 42 abutting the support part 52.

In embodiments in which the third hollow member 43 is not provided, the forwards movement of the second hollow member 42 may be regulated by the tip end of the second hollow member 42 abutting the inner wall surface 54b.

According to some embodiments, the forwards movement of the second hollow member 42 may be halted by part of the inner wall of the third hollow member 43. Specifically, a tapered shape should be formed so that the inner diameter of the third hollow member 43 contracts towards the tip end, or a protrusion or the like should be formed in the cavity of the third hollow member 43. Moreover, if the forwards movement of the second hollow member 42 is regulated by the outer peripheral surface at the tip end side of the second hollow member 42 coming into abutment with part of the inner wall of the third hollow member 43, the movement regulating member 47 regulates only the backwards movement of the second hollow member 42.

Furthermore, the backwards movement of the second hollow member 42 may be halted by part of the outer wall of the first hollow member 41. Specifically, a tapered shape should be formed so that the outer diameter of the first hollow member 41 expands towards the base end, or a protrusion or the like should be formed on the outer periphery of the first hollow member 41.

Furthermore, the backwards movement of the second hollow member 42 may be halted by the tip end of the second hollow member 42 being stopped by the base end (inner side) of the third hollow member 43. Specifically, the tip end of the second hollow member 42 should project radially outwards and the base end of the third hollow member 43 should project radially inwards, and the projections should be formed in such a way as to be able to engage.

In accordance with embodiments in which the backwards movement of the second hollow member 42 is regulated by the inner peripheral surface at the base end side of the second hollow member 42 coming into abutment with the outer wall of the first hollow member 41, or by the tip end of the second hollow member 42 coming into abutment with the base end (inner side) of the third hollow member 43, the movement regulating member 47 regulates only the forwards movement of the second hollow member 42.

In other words, the movement regulating member 47 is not an essential component, and the decision as to whether it is provided depends on the configuration of the first hollow member 41, second hollow member 42, third hollow member 43, stopper 51a and stopper 51b.

Furthermore, the first hollow member 41, second hollow member 42 and third hollow member 43 allow the suture thread 1 to pass therethrough, so the inner diameters thereof are set so that the suture thread 1 is able to pass therethrough. Furthermore, the first hollow member 41 and the second hollow member 42 are able to pass through the suture thread clamping member 44 which will be described later. The outer diameters of the first hollow member 41 and the second hollow member 42 therefore have to be set to the extent that they can pass through the cut in the suture thread clamping member 44.

Instead of forming the tapered portion in the through-hole formed on the base end side of the housing 50 on the suture thread feed mechanism 40 side, as in in the illustrated embodiment, it is equally possible to make the base end of the first hollow member 41 project outwards of the housing 50. This makes it possible to lighten the burden of inserting the suture thread 1. However, as shown in FIGS. 1 to 3, the base end of the first hollow member 41 does not have to project outwards of the housing 50. Furthermore, if the base end side of the first hollow member 41 is funnel-shaped (a shape in which the wall surface grows gradually larger in diameter towards the base end), this makes it easier to insert the suture thread 1. Furthermore, the first hollow member 41 may be long enough to reach the base end of the housing 50, and a funnel-shaped member having an inner cavity may be fitted to the base end of the first hollow member 41.

In addition, the first hollow member 41, second hollow member 42 and third hollow member 43 should be hollow inside and may, for example, comprise a hollow columnar member (cylindrical member) or a prismatic member (rectangular cylinder) or the like. Furthermore, a cut (or incision) may be formed in part of the first hollow member 41, second hollow member 42 and third hollow member 43 so that the hollow parts thereof can communicate with the outside.

In addition, the third hollow member 43 also has the function of guiding the forwards and backwards movement of the second hollow member 42. The axial length of the third hollow member 43 should therefore be determined not only taking account of the length of the suture thread 1 which is to be fed out from the tip end of the suture thread introduction needle 70, but also taking account of the length of the second hollow member 42. That is to say, the axial length of the third hollow member 43 still allows part of the second hollow member 42 to be positioned inside the hollow part of the third hollow member 43 when the second hollow member 42 has moved as far as possible backwards.

The suture thread clamping member 44 is made of natural rubber or synthetic rubber, or a metal, etc., for example, and is formed with a cut which runs therethrough in the front-to-rear direction in the centre. When the first hollow member 41, suture thread 1 and second hollow member 42 are inserted into the cut formed in the suture thread clamping member 44, the first hollow member 41, suture thread 1 and second hollow member 42 can be clamped and held by the friction force between the surface of the cut and each member. For example, the suture thread clamping member 44 can clamp and hold any of the first hollow member 41, suture thread 1 and second hollow member 42 depending on the position thereof, which varies according to the movement of the operation part 10.

Furthermore, the suture thread clamping member 44 is moved forwards and backwards while it is accommodated in the suture thread feed part 45. The suture thread clamping member 44 can move forwards until the second abutment part 45a of the suture thread feed part 45 runs into the first abutment part 33 of the loop feed/return part 31, and can move backwards until the base end surface of the suture thread feed part 45 runs into the inner wall surface 57b at the base end side of the housing 50. The suture thread 1 is fed out as a result of being clamped by the suture thread clamping member 44. This means that the suture thread 1 is clamped and fed out by the suture thread clamping member 44 while the suture thread clamping member 44 is separated from the first hollow member 41 and is clamping the second hollow member 42.

The suture thread clamping member 44 may consist of an elastic element made of natural rubber or synthetic rubber etc., or a ring made of a relatively soft metal, such as copper, for example. If the suture thread clamping member 44 consists of an elastic element, the suture thread 1 can be clamped using the elastic force thereof. If the suture thread clamping member 44 consists of an element such as a metal ring, the suture thread 1 can be clamped by plastic deformation which occurs at the same time as the ring is separated from the first hollow member 41. Furthermore, the cut formed in the suture thread clamping member 44 does not have to be a simple cut, and includes a slit shape or a hole having a smaller inner diameter than the outer diameter of the suture thread 1, or similar.

The suture thread feed part 45 comprises: the second abutment part 45a, a side wall extension part 45b, a main body part 45c and a fitting part 45d, and it can move backwards and forwards inside the housing 50 in response to the movement of the operation part 10. It should be noted that the suture thread feed part 45 may, for example, comprise a hollow columnar member (cylindrical member) or a prismatic member (rectangular cylinder) or the like, and the fitting part 45d thereof should be formed in such a way that the suture thread clamping member 44 can be fitted therein. Moreover, a guide for moving the suture thread feed part 45 forwards and backwards in a straight line (a groove into which the suture thread feed part 45 can be fitted, for example) may be formed inside the housing 50. In this case, the guide which is formed should not produce any friction resistance with respect to the movement of the suture thread feed part 45.

The second abutment part 45a is provided at the tip end of the side wall extension part 45b, for example, and is formed in the shape of a protrusion which projects towards the loop feed/return mechanism 30. The tip end surface of the operation part 10 which has moved forwards abuts the base end surface of the second abutment part 45a, and the movement from the operation part 10 is transmitted thereto. The second abutment part 45a is then subjected to a forwards force and the suture thread feed part 45 moves forwards.

Forwards movement of the suture thread feed part 45 is regulated by abutment of the tip end surface of the main body part 45c of the suture thread feed part 45 with the movement regulating member 47 which has stopped moving due to abutment with the base end surface of the stopper 51a.

Meanwhile, backwards movement of the suture thread feed part 45 is produced by the tip end of the second abutment part 45a being pressed by the base end surface of the first abutment part 33 which is formed on the loop feed/return part 31, and this movement is regulated by the base end surface of the suture thread feed part 45 abutting the inner wall surface 57b. However, the suture thread feed part 45 can automatically move backwards due to the resilience of the second elastic member 46 which will be described later. It should be noted that the shape of the second abutment part 45a is not limited to a protrusion, it simply needs to be a shape which can abut the first abutment part 33 and the operation part 10. Furthermore, the forwards and backwards movement of the second abutment part 45a is guided by the groove 22 which guides the movement of the operation part 10.

The side wall extension part 45b is an extension of part of the outer wall of the main body part 45c towards the tip end. The side wall extension part 45b is positioned on the loop feed/return mechanism 30 side. Moreover, there is no particular limitation as to the axial length of the side wall extension part 45b, but it is determined to take account of the size of the housing 50 and the length of the suture thread 1 to be fed out from the tip end of the suture thread introduction needle 70. Furthermore, there is no particular limitation as to the thickness of the side wall extension part 45b, but it should be strong enough to withstand the force applied by the second abutment part 45a.

The main body part 45c has the fitting part 45d formed therein, and is also formed with a through-hole which allows the suture thread 1 to pass through backwards and forwards, by way of the fitting part 45d. The main body part 45c is disposed inside the housing 50 in such a way as to be able to move backwards and forwards. As described above, if a guide for moving the suture thread feed part 45 forwards and backwards in a straight line is formed inside the housing 50, the main body part 45c may be disposed on this guide. Furthermore, the suture thread feed part 45 is moved backwards until the base end surface of the main body part 45c abuts the inner wall surface 57b of the housing 50.

The fitting part 45d is formed in the main body part 45c and allows the suture thread clamping member 44 to be fitted therein. The movement of the suture thread clamping member 44 is restricted when it is fitted into the fitting part 45d. It should be noted that there is no particular limitation to the size of the fitting part 45d provided that it has a size which allows the suture thread clamping member 44 to be fitted therein.

The second elastic member 46 consists of an elastic member which is extendible in the front-to-rear direction (for instance, a spring (coil spring, air spring etc.) or a rubber element). The second elastic member 46 is disposed between the inner wall surface 54b and the movement regulating member 47 which is fixed to the second hollow member 42, and extends and contracts between the inner wall surface 54b and the movement regulating member 47. The second elastic member 46 causes the suture thread feed part 45 to move backwards by pressing the movement regulating member 47 using the resilience thereof. If the second elastic member 46 consists of a spring, the second elastic member 46 should be disposed in such a way as to wind around the outer periphery of the third hollow member 43 and the second hollow member 42.

The loop introduction needle 60 has a cavity and accommodates the loop forming part 80 in said cavity in such a way that it can move forwards and backwards. When the loop introduction needle 60 is fitted to the housing 50 the base end thereof opens inside the housing 50 in order to allow communication with the inside of the housing 50. The loop introduction needle 60 is fitted to the tip end of the housing 50 in such a way that the axial centre thereof is aligned with the axial centre of the loop feed/return mechanism 30. If the loop introduction needle 60 is fitted to the housing 50 in this way, the loop forming part 80 can be fed out and returned in a straight line. The loop introduction needle 60 should be made of a metal such as stainless steel, for example.

Furthermore, the loop introduction needle 60 has a cutting surface, at the tip end, for piercing the skin. The cutting surface may be formed by cutting the loop introduction needle 60 on a plane which obliquely intersects the axial centre thereof. The tip end opening of the loop introduction needle 60 should be oriented towards the suture thread introduction needle 70 so that the loop 82 of the loop forming part 80 extends in the direction of the suture thread introduction needle 70 in a reliable manner, as will be described later. It should be noted that the tip end side of the loop introduction needle 60 does not mean only the very tip of the loop introduction needle 60, it also includes the side surfaces in the tip-end region of the loop introduction needle 60.

Moreover, there is no particular limitation as to the shape of the loop introduction needle 60 provided that it can pierce the skin and allows the loop forming part 80 to be inserted therein. For example, a needle of outer diameter around 21-17 G (preferably 20-18 G) and length in a range of from about 70 to about 120 mm (preferably in a range of from 80 to 100 mm) may be used as the loop introduction needle 60. Furthermore, a bevelled part may be formed at the tip end of the loop introduction needle 60 so as not to cut the suture thread 1 when it is gripped. In addition, it should be possible to adjust the position of the tip end of the loop introduction needle 60 (the position relative to the suture thread 1 which has been fed out) through the position of the support part 52.

The suture thread introduction needle 70 has a cavity in which the suture thread 1 is inserted in such a way as to be able to move forwards and backwards. The base end of the suture thread introduction needle 70 communicates with the hollow part of the third hollow member 43 when the suture thread introduction needle 70 is fitted to the housing 50. The suture thread introduction needle 70 is fitted to the tip end of the housing 50 in such a way that the axial centre thereof is aligned with the axial centre of the suture thread feed mechanism 40. If the suture thread introduction needle 70 is fitted to the housing 50 in this way, the suture thread 1 can be fed out in a straight line. The suture thread introduction needle 70 may be formed from a metal such as stainless steel, for example. Moreover, the base end of the suture thread introduction needle 70 may function as the third hollow member 43.

Furthermore, the suture thread introduction needle 70 has a cutting surface, at the tip end, for piercing the skin. The cutting surface may be formed by cutting the suture thread introduction needle 70 on a plane which obliquely intersects the axial centre thereof. There is no particular limitation as to the orientation of the tip end opening of the suture thread introduction needle 70, but it preferably faces the direction of the loop introduction needle 60. It should be noted that the tip end side of the suture thread introduction needle 70 does not mean only the very tip of the suture thread introduction needle 70, it also includes the side surfaces in the tip-end region of the suture thread introduction needle 70.

Moreover, there is no particular limitation as to the shape of the suture thread introduction needle 70 provided that it can pierce the skin and allows the suture thread 1 to be inserted therein. For example, a needle of outer diameter around 21-17 G (preferably 20-18 G) and length around 70-120 mm (preferably 80-100 mm) may be used as the suture thread introduction needle 70. Furthermore, it should be possible to adjust the position of the tip end of the loop introduction needle 60 (the position relative to the loop 82 which has been fed out) through the position of the support part 53.

As described above, the loop introduction needle 60 and the suture thread introduction needle 70 are supported in parallel a prescribed distance apart at the tip end of the housing 50. The distance between the loop introduction needle 60 and the suture thread introduction needle 70 should be set at the length over which the patient's abdominal wall and internal organ wall are to be fixed by the suture thread 1 (in a range of from 10 mm to 30 mm, for example). The patient's abdominal wall and internal organ wall can be properly fixed by the suture thread 1 if the distance between the loop introduction needle 60 and the suture thread introduction needle 70 is within this range.

The loop forming part 80 is inserted into the loop introduction needle 60 in such a way as to be able to move forwards and backwards. The loop forming part 80 comprises: the base end fixing part 83 which is fixed to the tip end of the loop feed/return part 31; the loop 82 which is fixed to the tip end; and a rod-like shaft 81 which has a smaller outer diameter than the inner diameter of the loop introduction needle 60 and connects the base end fixing part 83 and the loop 82. The loop 82 is made of an elastic material and it reverts to an annular shape when it has been fed out from the tip end of the loop introduction needle 60 (when no external force is applied thereto), and changes to a substantially linear shape which can be accommodated inside the loop introduction needle 60 when it has not been fed out from the tip end of the loop introduction needle 60. The planar shape of the loop 82 when it has been fed out from the tip end of the loop introduction needle 60 (when no external force is applied thereto) should be an endless ring.

When the loop 82 has been fed out from the tip end of the loop introduction needle 60, it extends in the direction of the suture thread introduction needle 70 in such a way that the suture thread 1 which is fed out from the tip end of the suture thread introduction needle 70 passes through the inside of the loop 82. For example, the loop 82 may be fixed to the tip end of the rod-like shaft 81 at a prescribed angle so as to form a curved shape when it has been fed out from the tip end of the loop introduction needle 60. This curved shape may be such that when the loop 82 is seen from the side the top part of the curved section projects forwards, for example. If the loop 82 is formed with this kind of shape, the suture thread 1 can be positioned inside the loop 82 with greater certainty. Furthermore, if the centre of the loop 82 lies over the extension of the suture thread introduction needle 70, then when the suture thread 1 extends straight forwards, said suture thread 1 can be positioned inside the loop 82 with greater certainty.

Furthermore, although there is no particular limitation to the tip end shape of the loop 82, it may be substantially V-shaped or substantially U-shaped with the tip end at the centre in order to narrow the distance between the sections which grip the suture thread 1 (the substantially V-shaped or substantially U-shaped portion). If this kind of shape is adopted, the suture thread 1 which has been fed out from the tip end of the suture thread introduction needle 70 can be gripped more securely. The loop 82 may consist of a deformable member, for example: stainless steel wire (high tensile stainless steel for springs); piano wire (nickel-plated or chromium-plated piano wire); or superelastic alloy wire (titanium-nickel alloy, copper-zinc alloy (or an alloy containing beryllium, silicon, tin, aluminium or gallium etc. therewith), nickel-aluminium alloy etc.).

The rod-like shaft 81 may be constructed using, among other things, a metal (e.g., stainless steel) or a synthetic resin (e.g., a polyolefin such as polypropylene or polyethylene, or a fluororesin such as PTFE or ETFE). A stylet or the like may be used for the rod-like shaft 81. Furthermore, provided that the base end fixing part 83 is fixed to the tip end of the loop feed/return part 31, there is no particular limitation to the method of fixing the base end fixing part 83. For example, the base end fixing part 83 may be fixed using adhesive or the like, or a notch etc. may be provided at the tip end of the loop feed/return part 31 and the base end fixing part 83 may be fixed thereto by fitting into said notch etc. Moreover, when the loop 82 is made of a relatively rigid material, the rod-like shaft 81 and the loop 82 may be made of the same material. If that is the case, the rod-like shaft 81 and the loop 82 may be formed as a single element or as separate elements.

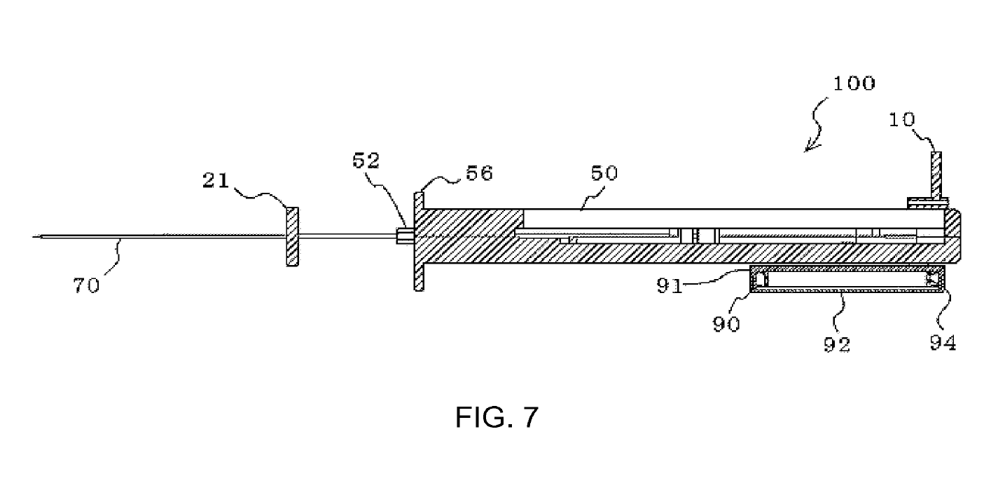

FIG. 7 is a schematic view in cross section along A-A in FIG. 4. The thread housing part 90 will be described below with reference to FIGS. 1-5 and 7. It should be noted that FIG. 7 focuses on the configuration associated with the thread housing part 90.

The thread housing part 90 is a member for accommodating the suture thread 1 on the upstream side in the feed-out direction of the suture thread 1 which is supplied to the suture thread feed mechanism 40 (i.e., upstream in the direction of advance when the suture thread 1 is fed out). In the illustrated embodiment, the thread housing part 90 is provided on the opposite side of the housing 50 to the side where the operation part 10 is provided. The thread housing part 90 comprises a housing part 91, substantially circular in plan view, which opens on the upper surface and comprises an internal housing space; a cover 92 for covering the opening in the upper surface of the housing part 91; and a bobbin 94 which is housed inside the housing part 91. The housing part 91, cover 92 and bobbin 94 are made of a synthetic resin material, for example, a thermoset plastic. Other material can be used for example, ABS or PVC plastic.

According to some embodiments, the housing part 91 is detachably fitted to the housing 50. This enables the thread housing part 90 to be replaced on the housing 50. It should be noted that the housing part 91 and the housing 50 may be moulded as a single piece.

In further embodiments, the cover 92 is detachable from the housing part 91. This makes it possible, for example, to straighten tangling of the suture thread 1 if the suture thread 1 is tangled while housed inside the housing part 91, and to accommodate a different suture thread 1 inside the housing part 91. The structure used for fixing the cover 92 and the housing part 91 may be, for example, a screw structure, a bottle cap-type structure, a latching structure employing hooks, or any structure, but a structure with which the cover 92 does not readily fall off during use is preferable.

Furthermore, the cover 92 is preferably made of a transparent or translucent material. This enables the practitioner to ascertain the remaining amount of suture thread 1 or the state of the suture thread 1 through the cover 92, which makes the instrument easier to use.

A thread dispensing opening 93 is formed in the thread housing part 90 in order to dispense the suture thread 1 housed therein. According to the illustrated embodiment, part of the peripheral wall of the housing part 91 is cut away in order to form the thread dispensing opening 93. Moreover, part of the peripheral wall of the cover 92 rather than the peripheral wall of the housing part 91 may equally be cut away in order to form the thread dispensing opening 93. Furthermore, the corresponding peripheral walls of the housing part 91 and cover 92 when the cover 92 is closed may be cut away, respectively, in order to form an opening, and when the cover 92 is closed, the openings provided in the housing part 91 and cover 92 form a single opening which constitutes the thread dispensing opening 93.

As shown in FIGS. 4 and 5 etc., the thread dispensing opening 93 opens at a position in the opposite direction to the thread insertion opening 58a from the widthwise centre of the housing 50. That is to say, the thread insertion opening 58a is provided towards the suture thread feed mechanism 40 in the widthwise (short) direction of the housing 50, whereas the thread dispensing opening 93 is provided towards the loop feed/return mechanism 30 in the widthwise direction of the housing 50. This means that the tangent to the thread housing part 90 at the thread dispensing opening 93 is close to an imaginary line which joins the thread dispensing opening 93 and the thread insertion opening 58a. Consequently, the suture thread 1 running from the thread dispensing opening 93 to the thread insertion opening 58a is unlikely to become bent or curved, the suture thread can be withdrawn smoothly, and it is possible to restrict damage to the suture thread 1 caused by bending or folding.

In accordance with the illustrated embodiment, there is no covering member for the suture thread 1 provided for at least part of the suture thread 1 running from the thread dispensing opening 93 to the thread insertion opening 58a. That is to say, the suture thread 1 which has been withdrawn from the thread dispensing opening 93 is at least partly exposed over a range up to the thread insertion opening 58a. By making the suture thread 1 exposed in this way between the thread dispensing opening 93 and the thread insertion opening 58a it is possible for the practitioner to grip the exposed section of the suture thread 1 in order to push or pull it. According to other embodiments, a means for covering the suture thread 1 may be provided so that it is not exposed between the thread dispensing opening 93 and the thread insertion opening 58a, and in this way the practitioner's hand or an implement will not catch on the suture thread 1 during the procedure.

In some embodiments, the bobbin 94 serves as an element on which the suture thread 1 is wound, and is housed inside the housing part 91 in such a way as to be detachable and rotatable in the peripheral direction.

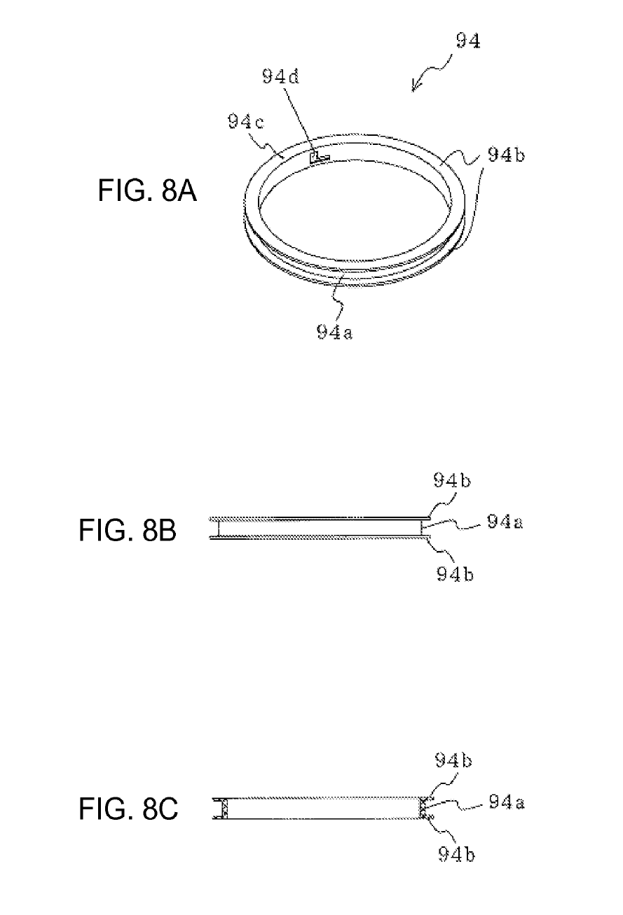

FIGS. 8A-8C illustrate the structure of the bobbin 94 according to some embodiments, where FIG. 8A is an oblique view of the bobbin 94, FIG. 8B is a side view of the bobbin 94, and FIG. 8C is a view in cross section of the bobbin 94. As shown in FIGS. 8A-8C, the bobbin 94 comprises a substantially cylindrical thread winding part 94a, and a flange part 94b which is provided at both ends of the thread winding part 94a and projects radially outwards from the thread winding part 94a. The suture thread 1 is wound in the circumferential direction onto the outer circumference of the winding part 94a. By providing this kind of bobbin 94, it is possible to restrict tangling of the suture thread 1 inside the thread housing part 90.

If the outer diameter of the thread winding part 94a of the bobbin 94 is excessively large, the outer diameter of the thread housing part 90 also increases and it may obstruct the practitioner as he or she operates the suturing instrument 100. On the other hand, if the outer diameter of the thread winding part 94a is excessively small, the suture thread 1 which has been wound has a strong tendency to retain its wound shape, so the suture thread 1 can no longer be inserted into the loop 82 in the loop forming part 80 when the suture thread 1 is fed out from the tip end of the suture thread introduction needle 70, as will be described later. The outer diameter of the thread winding part 94a of the bobbin 94 should therefore be set in such a way as to take account of handling by the practitioner and the tendency of the suture thread 1 to retain its wound shape.

Furthermore, the external shape of the thread winding part 94a of the bobbin 94 is not limited to a substantially circular shape, and an elliptical shape or rectangular shape with rounded corners, or a polygonal shape is equally feasible. Moreover, if the external shape of the thread winding part 94a is elliptical, there is a large amount of curvature in the suture thread 1 in the region of the long diameter part, if the external shape of the thread winding part 94a is a rectangle with rounded corners, there is a large amount of curvature in the suture thread 1 in the region of the corners, and if the external shape of the thread winding part 94a is polygonal, there is a large amount of curvature in the suture thread 1 in the region of the angled parts, so the external shape of the thread winding part 94a should be designed in such a way that the suture thread 1 does not readily tend to retain its wound shape in the portions which are subject to a large amount of curvature.

Moreover, it is also possible to provide a means for removing the curls produced in the suture thread 1, for instance by stretching. By providing such a means, the suture thread 1 is easily inserted into the loop 82 of the loop forming part 80 when it is fed out from the suture thread introduction needle 70 even if the suture thread 1 tends to retain its wound shape.

The flange part 94b suppresses incidences of the suture thread 1 being dislodged from the two end parts of the thread winding part 94a. It should be noted that a configuration without the flange part 94b is also possible.

Furthermore, a notch 94c which can catch the suture thread 1 is formed in part of the flange part 94b. If the tip end portion of the suture thread 1 (the tip end portion at the end of the winding on the thread winding part 94a) is caught and temporarily fixed in the notch 94c, it is possible to suppress unravelling of the suture thread 1 from the thread winding part 94a.

Furthermore, a thread stop hole 94d which runs through the thread winding part 94a is formed in the thread winding part 94a. The thread stop hole 94d is intended to catch the tip end portion of the inserted suture thread 1 at the start of winding. In accordance with the illustrated embodiment, the thread stop hole 94d is formed as a hook shape, but any shape may be adopted for the thread stop hole 94d provided that it is capable of securing the suture thread 1. By providing the thread stop hole 94d, the suture thread 1 does not readily become detached when it is wound on the thread winding part 94a, and the wound suture thread 1 does not readily become dislodged from the thread winding part 94a.

As shown in FIG. 4, the bobbin 94 on which the suture thread 1 has been wound is preferably accommodated inside the housing part 91 at an orientation such that the withdrawal direction of the suture thread 1 at the thread dispensing opening 93 and an imaginary line joining the thread dispensing opening 93 and the thread insertion opening 58a are substantially the same direction. For example, if the bobbin 94 were accommodated inside the housing part 91 in the opposite way to the example shown in FIG. 4, there would be a large amount of curvature in the suture thread 1 at the thread dispensing opening 93 and the suture thread 1 could be damaged by friction with the thread dispensing opening 93. However, when the bobbin 94 is accommodated in the housing part 91 at the orientation shown in FIG. 4, the suture thread 1 which has been withdrawn from the thread dispensing opening 93 is conducted to the thread insertion opening 58a with essentially no curvature. Moreover, a mark indicating the orientation (direction of winding of the suture thread 1) when the bobbin 94 is accommodated inside the housing part 91 may be provided on the housing part 91 or cover 92, and by doing so it is unlikely that the practitioner will place the bobbin 94 at the wrong orientation.

The position where the thread housing part 90 is fitted to the housing 50 should be determined to take account of the centre of gravity of the suturing instrument 100 and the position where the practitioner holds the suturing instrument 100. For example, when the thread housing part 90 is provided at the tip end side of the housing 50 and the suturing instrument 100 is placed upright in such a way that the tip ends of the loop introduction needle 60 and the suture thread introduction needle 70 are directed towards the patient, the centre of gravity is at the lower side (patient side), so there is increased stability when the practitioner supports the suturing instrument 100, but at the same time the thread housing part 90 may also obstruct the practitioner's grip of the suturing instrument 100. On the other hand, when the thread housing part 90 is provided towards the base end of the housing 50 and the suturing instrument 100 is placed upright in the same way, the thread housing part 90 is unlikely to obstruct the practitioner's grip of the suturing instrument 100, but the centre of gravity of the suturing instrument 100 is at the upper side, so there may be reduced stability when the suturing instrument 100 is supported. The position where the thread housing part 90 is fitted should therefore be determined to take account of stability when the suturing instrument 100 is placed upright and the ease with which the practitioner can grip the suturing instrument 100.

Furthermore, in accordance some embodiments, the thread housing part 90 is provided above the housing 50, but the position where the thread housing part 90 is provided is not limited to this position. An alternate embodiment in which the thread housing part 90 is fitted to the housing 50 in one position will be described below.

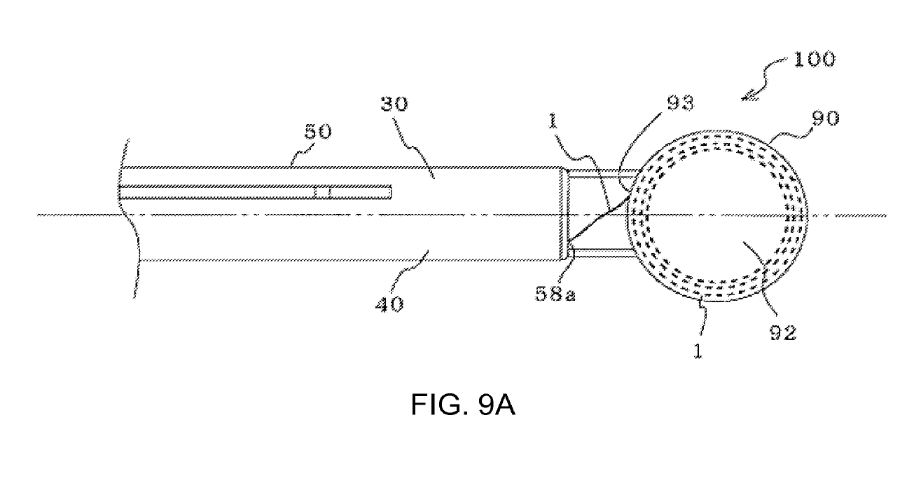

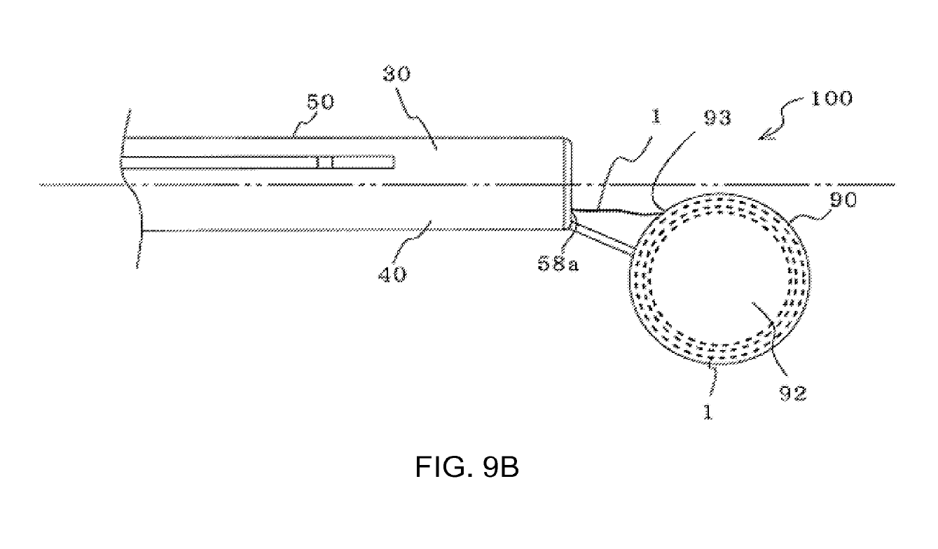

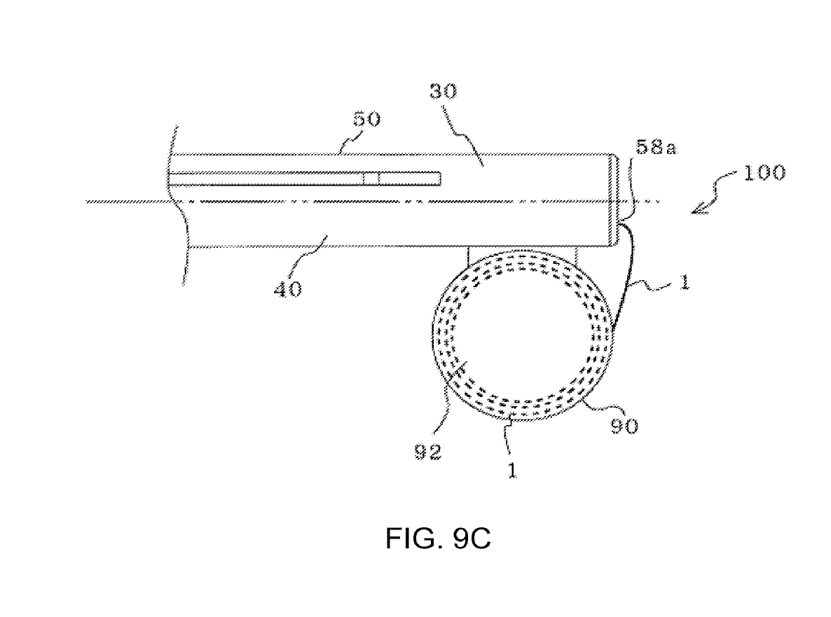

FIGS. 9A-9C illustrates examples of the fitting position of the thread housing part 90 according to one embodiment. The main structural constituents of the thread housing part are the further illustrated and described with reference to FIGS. 9A-9C.

FIG. 9A shows an example in which the thread housing part 90 is fitted to the housing 50 by way of a linking member further towards the base end side than the base end of the housing 50. FIG. 9B shows an example in which the thread housing part 90 is fitted to the housing 50 by way of a linking member further towards the base end side than the base end of the housing 50 and at a position approaching the suture thread feed mechanism 40 side. FIG. 9C shows an example in which the thread housing part 90 is fitted to the housing 50 by way of a linking member at a position beside the suture thread feed mechanism 40 side of the housing 50. Furthermore, in the examples shown in FIGS. 9A-9C, the thread insertion opening 58a is positioned substantially in the direction directly ahead of the suture thread 1 at the thread dispensing opening 93 so that bending and curvature of the suture thread 1 as it arrives at the thread insertion opening 58a from the thread dispensing opening 93 is prevented as far as possible. In addition to the examples shown in FIG. 9A-9C, the position where the thread housing part 90 is fitted to the housing 50 may be determined taking account of ease of handling by the practitioner and the path of the suture thread 1 as it arrives at the thread insertion opening 58a from the thread dispensing opening 93, among other things. Furthermore, the thread housing part 90 may be directly fitted to the housing 50 without the interposition of another member like the linking member shown in the examples in FIGS. 9A-9C.

According to some embodiments, in the suturing instrument 100, the operation part 10 and the loop feed/return mechanism 30 and suture thread feed mechanism 40 are not linked. In further embodiments, the loop feed/return mechanism 30 and the suture thread feed mechanism 40 are not linked either. That is to say, the operation part 10, loop feed/return mechanism 30 and suture thread feed mechanism 40 are independent of one another in accordance with these embodiments. Nonetheless, a simple operation of the suturing instrument 100 makes it possible to provide a time delay between the timing for feeding out the loop 82 and the timing for feeding out the suture thread 1, without requiring excessive work by the practitioner for operation, and a situation can be achieved in which the suture thread 1 passes through the internal organ wall and the abdominal wall. The procedure for achieving this is described in more detail, below.

As a preliminary operation of the suturing instrument 100, the practitioner first of all withdraws one end of the suture thread 1 inside the thread housing part 90 from the thread dispensing opening 93, and inserts the suture thread 1 into the first hollow member 41 of the suture thread feed mechanism 40 through the thread insertion opening 58a. When the suture thread 1 inside the thread housing part 90 is pulled out from the thread dispensing opening 93, the bobbin 94 inside the thread housing part 90 rotates in the circumferential direction, so the suture thread 1 which is wound on the bobbin 94 is smoothly fed out. At this point, the practitioner feeds the suture thread 1 into the suture thread introduction needle 70 to a length of such an extent that the suture thread 1 does not protrude from the tip end of the suture thread introduction needle 70. The suture thread 1 is exposed over the region from the thread dispensing opening 93 to the thread insertion opening 58a, so the practitioner can hold the exposed portion of the suture thread 1 in order to push or pull the suture thread 1. When the operation part 10 is moved forwards after the suturing instrument 100 has been made to pierce into the internal organ, the forwards movement of the operation part 10 is first of all transmitted to the loop feed/return mechanism 30, and is then transmitted to the suture thread feed mechanism 40. It should be noted that the practitioner may make use of an assembly in which the suture thread 1 is already set in the suturing instrument 100.

In the initial state (or the state prior to the second and subsequent operations) in which the operation part 10 is not being operated, the situation on the loop feed/return mechanism 30 side is that the base end part 31b of the loop feed/return part 31 is abutting the inner wall surface 57a of the housing 50. Here, the first elastic member 32 which is being compressed by a prescribed force is accommodated inside the accommodation part 34. Furthermore, the operation part 10 is held between the first elastic member 32 and the base end part 31b of the loop feed/return part 31.

When the practitioner applies an input operation to the operation part 10 so that the operation part 10 is moved forwards, this force is transmitted to the first elastic member 32. The first elastic member 32 is compressed by a prescribed force, so the tip end part 31a of the loop feed/return part 31 is pushed by the tip end of the first elastic member 32. That is, the operation part 10, loop feed/return part 31 and first elastic member 32 move forwards while the respective positions thereof are maintained. When this happens, the loop forming part 80 fixed to the tip end part 31a of the loop feed/return part 31 also moves forwards, and the loop 82 positioned at the tip end of the loop forming part 80 juts out from the tip end of the loop introduction needle 60, and opens into an annular shape.

When the operation part 10 is moved further forwards, the first abutment part 33 of the loop feed/return part 31 runs into the inner wall surface 54b. In this state, when a force greater than the force being applied to the first elastic member 32 is transmitted to the first elastic member 32 by way of the operation part 10, the first elastic member 32 is further compressed. That is, the loop 82 which has been fed out from the tip end of the loop introduction needle 60 can be maintained in an open state. By virtue of this configuration, it is possible to adjust the timing for feeding out the suture thread 1 and the timing for feeding out the loop 82, for instance delaying the timing for feeding out the suture thread 1 with respect to the timing for feeding out the loop 82. In other words, the annular shape of the loop 82 can be maintained as the operation part 10 continues to move forwards.

Next, when the operation part 10 is no longer being operated towards the tip end, the compressive force acting on the first elastic member 32 is released, and the loop feed/return part 31 starts to move backwards. That is, the operation part 10 moves backwards under the resilience of the first elastic member 32 until it abuts the base end part 31b of the loop feed/return part 31. When the practitioner then moves the operation part 10 further backwards, the rearward force of the operation part 10 which is abutting the base end part 31b of the loop feed/return part 31 is transmitted to the loop feed/return part 31. As a result, the base end part 31b of the loop feed/return part 31 is pushed backwards and the loop feed/return part 31 moves backwards.

A separate elastic element such as a spring or a rubber element may be provided in addition to the first elastic member 32, and the operation part 10 may be moved backwards using the resilience of this elastic element. When this kind of elastic element is provided, the operation to move the operation part 10 backwards is simplified, and the work of the practitioner can be lightened. That is, the suture thread 1 can be inserted through the internal organ wall and the abdominal wall essentially by means of a one-touch operation, without the practitioner having to perform a return operation, as will be described later.

When the loop feed/return part 31 moves backwards, the loop 82 also starts to move backwards. At this point, the suture thread 1 which has been fed out from the tip end of the suture thread introduction needle 70 has passed through the loop 82. When the loop 82 starts to move backwards, the annular shape of the loop 82 therefore becomes gradually smaller, and the suture thread 1 is gripped by the loop 82. As the loop 82 moves further backwards, the suture thread 1 is gripped at the tip end of the loop introduction needle 60.

In the initial state (or the state prior to the second and subsequent operations) in which the operation part 10 is not being operated, the situation on the suture thread feed mechanism 40 side is that the suture thread clamping member 44 has over-mounted the first hollow member 41, which is to say that the first hollow member 41 is clamped, so the suture thread 1 is not held by the suture thread clamping member 44 and is in a free state of mobility (a state in which the suture thread 1 is not clamped by the suture thread clamping member 44 and can be freely advanced and retracted).

Even if the practitioner moves the operation part 10 forwards, operation does not start straight away on the suture thread feed mechanism 40 side. In addition, the suture thread feed part 45 starts to move forwards when the operation part 10 is moved forwards and abuts the second abutment part 45a. That is, the movement of the operation part 10 is transmitted to the components of the suture thread feed mechanism 40 by way of the second abutment part 45a.

When the suture thread feed part 45 moves forwards, the suture thread clamping member 44 also moves forward in tandem. When the suture thread clamping member 44 moves by a prescribed distance, the first hollow member 41 is withdrawn from the suture thread clamping member 44. The suture thread 1 is then in a state in which it is running through the cut in the suture thread clamping member 44, in other words a state in which the suture thread clamping member 44 is clamping the suture thread 1. In this state, the clamping force of the suture thread clamping member 44 is transmitted to the suture thread 1 and the suture thread 1 is gripped by the suture thread clamping member 44.

At this point, the base end of the second hollow member 42 is in contact with the tip end of the suture thread clamping member 44. The movement of the operation part 10 is then transmitted to the second hollow member 42 by way of the suture thread clamping member 44, and the second hollow member 42 also moves forwards as it is pushed by the suture thread clamping member 44. Moreover, the forwards movement of the second hollow member 42 causes the second elastic member 46 to be pushed by the movement regulating member 47, so the second elastic member 46 is compressed in the forwards direction.

The suture thread 1 is gripped by the suture thread clamping member 44 and is therefore fed out from the tip end of the suture thread introduction needle 70 together with the forwards movement of the second hollow member 42. That is to say, the suture thread 1 is fed out from the tip end of the suture thread introduction needle 70 at a delayed timing with respect to the timing at which the loop 82 is fed out from the tip end of the loop introduction needle 60. This means that it is possible to provide a time delay between the timing for feeding out the loop 82 and the timing for feeding out the suture thread 1, and the suture thread 1 can be easily inserted into the loop 82. Moreover, the suturing instrument 100 makes it possible to freely adjust the timing for feeding out the suture thread 1 and the timing for feeding out the loop 82.

When the operation part 10 is moved further forwards, the movement regulating member 47 runs into the stopper 51a. When this occurs, the forwards movement of the suture thread feed part 45 together with the second hollow member 42 is stopped. In this state, a greater force than the force which is being applied to the first elastic member 32 is transmitted to the first elastic member 32 by way of the operation part 10, and when the operation part 10 is moved further forwards, the suture thread feed part 45 moves forwards whereby the base end of the second hollow member 42 penetrates the cut in the suture thread clamping member 44 and is held. When this occurs, the clamping of the suture thread 1 by the suture thread clamping member 44 is released and the suture thread 1 is in a free state. That is, the suture thread 1 does not move forwards.

Next, when operation of the operation part 10 towards the tip end is halted, the compressive force acting on the second elastic member 46 is released and the suture thread feed part 45 moves backwards as far as the position where the movement regulating member 47 runs into the stopper 51b. However, the second hollow member 42 remains penetrating and held by the suture thread clamping member 44.

When the operation part 10 is moved further backwards while the movement regulating member 47 is in abutment with the stopper 51b, the base end surface of the first abutment part 33 of the loop feed/return part 31 abuts the tip end surface of the second abutment part 45a of the suture thread feed part 45, and the force of the operation part 10 is transmitted to the suture thread feed part 45 by way of the loop feed/return part 31. The movement of the second hollow member 42 is stopped by the stopper 51b, and therefore only the suture thread feed part 45 moves backwards. The second hollow member 42 is withdrawn from the cut in the suture thread clamping member 44, which then clamps the first hollow member 41. That is to say, the clamping force of the suture thread clamping member 44 stops being applied to the second hollow member 42 and is applied to the first hollow member 41.

As will be described later, with the suturing instrument 100, the feeling of the suture thread 1 being gripped at the tip end of the loop introduction needle 60 is directly transmitted to the practitioner, so when the suture thread 1 is gripped at the tip end of the loop introduction needle 60, the components (operation part 10, loop feed/return mechanism 30 and suture thread feed mechanism 40) have not yet returned to their initial state. That is to say, while the suture thread 1 is being gripped by the loop introduction needle 60, the loop forming part 80 cannot enter the loop introduction needle 60 and the loop feed/return part 31 cannot move any further backwards.

When the loop introduction needle 60 and suture thread introduction needle 70 of the suturing instrument 100 are withdrawn from inside the internal organ to the abdominal wall side while the suture thread 1 is being gripped at the tip end of the loop introduction needle 60, the suture thread 1 is smoothly guided because it is free, and the suture thread 1 assumes a state of having passed through the internal organ wall and the abdominal wall in substantially a U-shape. Next, the suture thread 1 is cut using the cutting surface of the suture thread introduction needle 70 which has been withdrawn to outside the body, or another cutting means (scissors, cutter etc.). The excess suture thread 1 gripped at the tip end of the loop introduction needle 60 is then removed. When this is done, the loop forming part 80 is able to enter the loop introduction needle 60, and the loop feed/return part 31 can move backwards. In this state, if the operation part 10 is moved further backwards, the first abutment part 33 and second abutment part 45a come into abutment, and each component returns to the same position as in the initial state. Accordingly, the same state as the initial state is reached, in which the suture thread 1 is inserted up to the tip end of the suture thread introduction needle 70, and therefore the internal organ wall can be fixed any number of times using the above procedure. Moreover, if the suture thread 1 is projecting further towards the tip end than the cutting edge of the suture thread introduction needle 70 after the suture thread 1 has been cut, the same state as the initial state may be set by pulling the suture thread 1 towards the base end by hand.

However, the first abutment part 33 and second abutment part 45a may be brought into abutment at the same time as the suture thread 1 is gripped at the tip end of the loop introduction needle 60.

By virtue of the suturing instrument 100, when an input operation is applied to the operation part 10 by the practitioner, the loop feed/return mechanism 30 operates in such a way that the loop 82 projects from the tip end of the loop introduction needle 60, and after at least part of the loop 82 has projected from the tip end of the loop introduction needle 60, the suture thread feed mechanism 40 operates in such a way that the suture thread 1 projects from the tip end of the suture thread introduction needle 70.

To be specific, when the operation part 10 is moved towards the tip end, the operating force on the operation part 10 is transmitted to the loop feed/return mechanism 30, the loop feed/return mechanism 30 starts to act first of all and only the loop 82 is fed out at an early stage using the elastic force of the first elastic member 32. Then, when the loop 82 has been completely fed out (the loop 82 is restored to an annular shape), the movement of the operation part 10 is transmitted only to the first elastic member 32 on the loop feed/return mechanism 30 side in such a way that the suture thread 1 is fed out, and the shape of the loop 82 can be maintained. However, the timing for feeding out the suture thread 1 and the timing for feeding out the loop 82 can be adjusted so that the suture thread 1 may be fed out without the loop 82 being completely fed out. Furthermore, the suture thread 1 and loop 82 may be fed out at the same time.

Meanwhile, the suture thread feed mechanism 40 starts operating at a later stage than the operation of the loop feed/return mechanism 30. A predetermined time after the start of operation, the suture thread feed mechanism 40 assumes a free state in which the suture thread clamping member 44 over-mounts the first hollow member 41 and no force from the suture thread feed mechanism 40 is applied to the suture thread 1. In the suture thread feed mechanism 40, the suture thread clamping member 44 then clamps the suture thread 1 by withdrawing from the first hollow member 41, and the movement of the operation part 10 can be transmitted to the suture thread 1.

In this way, the suturing instrument 100 makes it possible to provide a time delay between the timing for feeding out the loop 82 and the timing for feeding out the suture thread 1. The suture thread 1 starts to be fed out once at least part of the loop 82 is projecting from the tip end of the loop introduction needle 60, so the suture thread 1 is readily inserted into the loop 82 which is formed into an annular shape. More preferably, the suture thread 1 may start to be fed out once the loop 82 has been fed out from the loop introduction needle 60, and by this means, the suture thread 1 can be reliably inserted into the loop 82 and the suture thread 1 can be firmly gripped. The suturing instrument 100 makes it possible to alter and correct the timing for feeding out the suture thread 1 and the timing for feeding out the loop 82 by adjusting the lengths of the loop feed/return part 31, first hollow member 41 and second hollow member 42, and the positions of the stopper 51a and stopper 51b.

Shortly after the loop feed/return part 31 has been moved backwards, the first abutment part 33 of the loop feed/return part 31 abuts the second abutment part 45a of the suture thread feed part 45. When the first abutment part 33 of the loop feed/return part 31 comes into abutment with the second abutment part 45a of the suture thread feed part 45, the feeling of this abutment is transmitted to the practitioner by way of the operation part 10. If the suture thread 1 is gripped by the loop 82 after the first abutment part 33 of the loop feed/return part 31 has come into abutment with the second abutment part 45a of the suture thread feed part 45, it is difficult to judge whether the suture thread 1 is being gripped by the loop 82. If the suture thread 1 is gripped by the loop 82 before the first abutment part 33 of the loop feed/return part 31 comes into abutment with the second abutment part 45a of the suture thread feed part 45, it is a simple matter to judge the state of gripping of the suture thread 1 by the loop 82, and the ease of use for the practitioner is further improved.

With the suturing instrument 100, the base end surface of the first abutment part 33 and the tip end surface of the second abutment part 45a are designed to come into abutment after the suture thread 1 has been gripped at the tip end of the loop introduction needle 60 or at the same time as the suture thread 1 is gripped at the tip end of the loop introduction needle 60. As a result, the feeling of the suture thread 1 being gripped at the tip end of the loop introduction needle 60 is transmitted directly to the practitioner by way of the housing 50 through the operation part 10. Moreover, the timing at which the base end surface of the first abutment part 33 and the tip end surface of the second abutment part 45a come into abutment should be at the same time as the suture thread 1 is gripped at the tip end of the loop introduction needle 60 or after the suture thread 1 has been gripped at the tip end of the loop introduction needle 60. It is therefore a simple matter for the practitioner to judge that the suture thread 1 has been gripped at the tip end of the loop introduction needle 60, and the ease of use is further improved.

The suturing instrument 100 is formed with a clearance such that the base end surface of the first abutment part 33 and the tip end surface of the second abutment part 45a do not come into abutment when the suture thread 1 is gripped at the tip end of the loop introduction needle 60. Furthermore, the suturing instrument 100 is such that the base end surface of the first abutment part 33 and the tip end surface of the second abutment part 45a come into abutment when the suture thread 1 is gripped at the tip end of the loop introduction needle 60. That is to say, by adjusting the distance between the base end surface of the first abutment part 33 and the tip end surface of the second abutment part 45a, it is possible to adjust the timing when the suture thread 1 is gripped at the tip end of the loop introduction needle 60 and the timing of abutment between the base end surface of the first abutment part 33 and the tip end surface of the second abutment part 45a. As a result, the timing of abutment between the base end surface of the first abutment part 33 and the tip end surface of the second abutment part 45a can be delayed with respect to the timing when the suture thread 1 is gripped at the tip end of the loop introduction needle 60, or can be synchronized with the timing when the suture thread 1 is gripped at the tip end of the loop introduction needle 60.

Moreover, when the timing of abutment between the base end surface of the first abutment part 33 and the tip end surface of the second abutment part 45a can be delayed with respect to the timing when the suture thread 1 is gripped at the tip end of the loop introduction needle 60, a clearance should be formed between the base end surface of the first abutment part 33 and the tip end surface of the second abutment part 45a when the suture thread 1 is gripped at the tip end of the loop introduction needle 60, and this clearance is not limited to a prescribed value. For example, the clearance should be adjusted to a value such that the base end surface of the first abutment part 33 and the tip end surface of the second abutment part 45a do not come into abutment at least until the tip end of the loop 82 is substantially coincident with the tip end of the loop introduction needle 60.

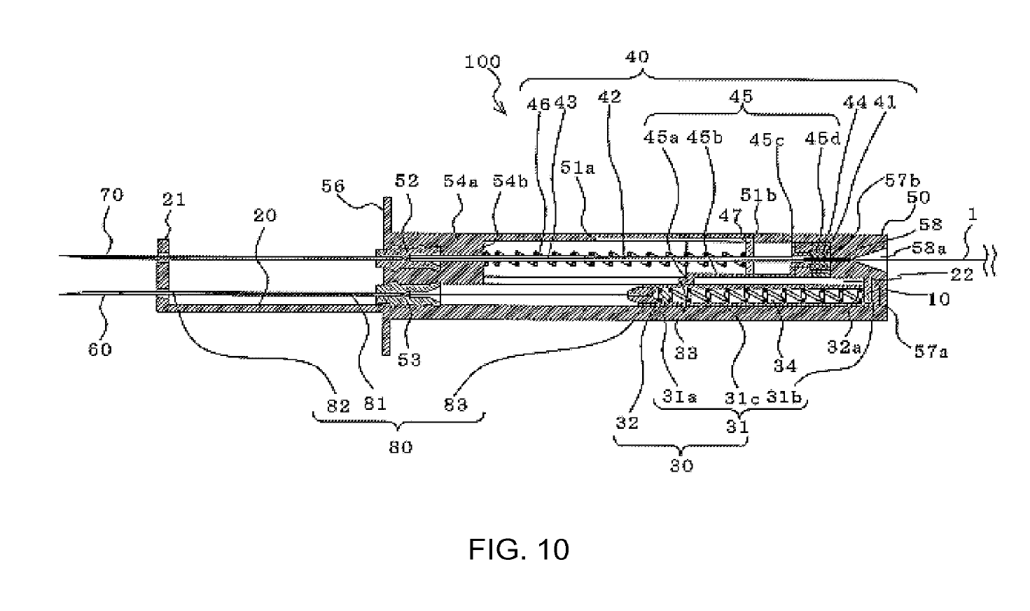

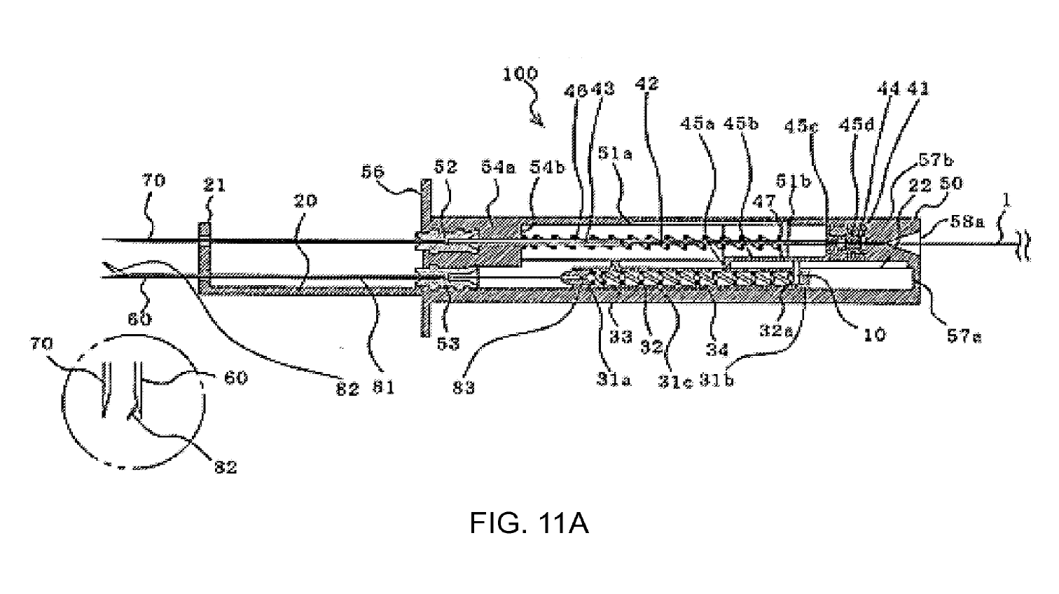

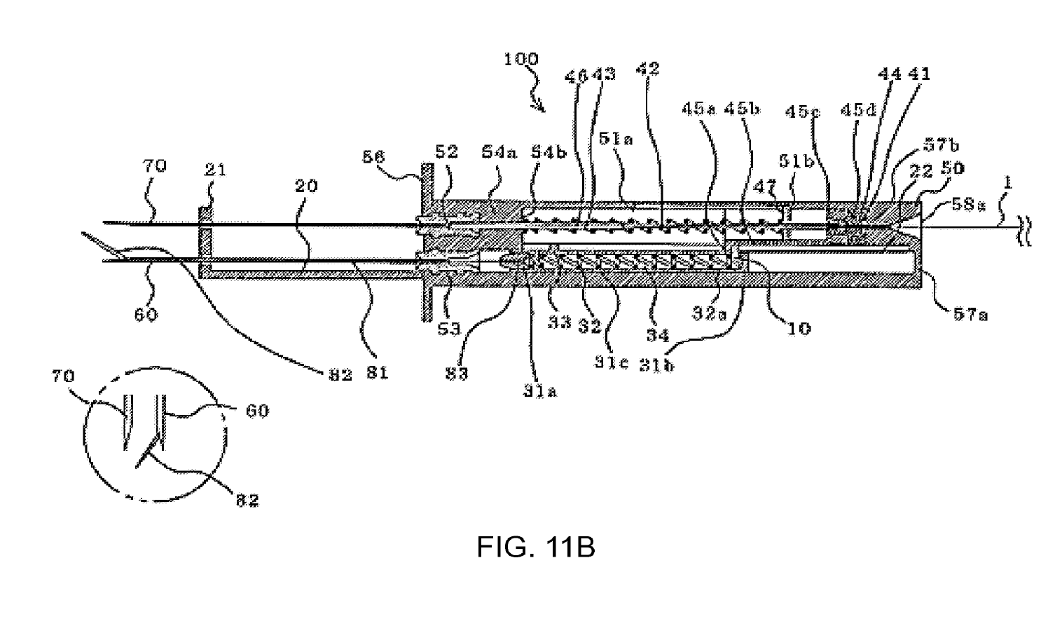

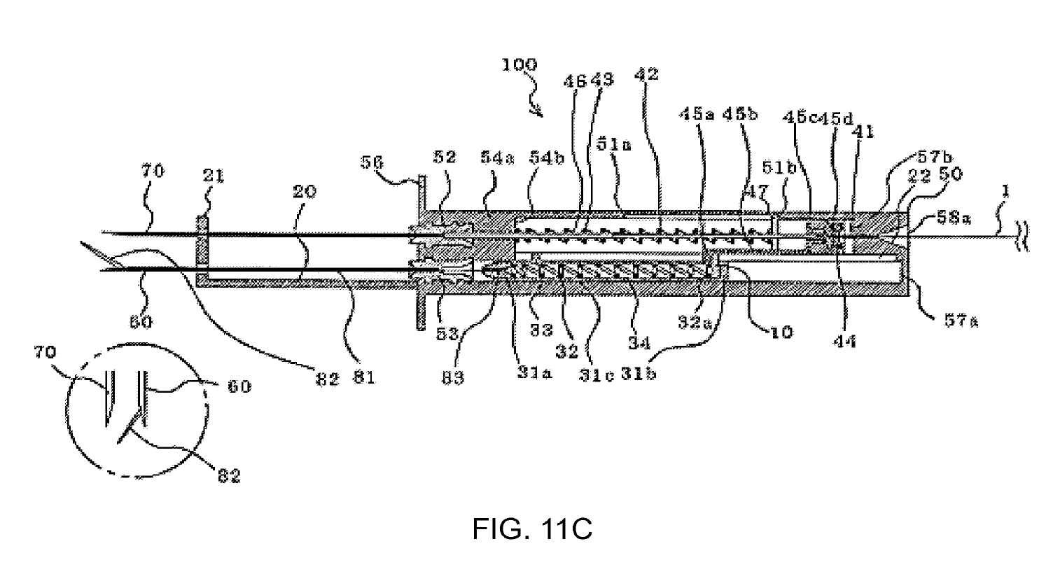

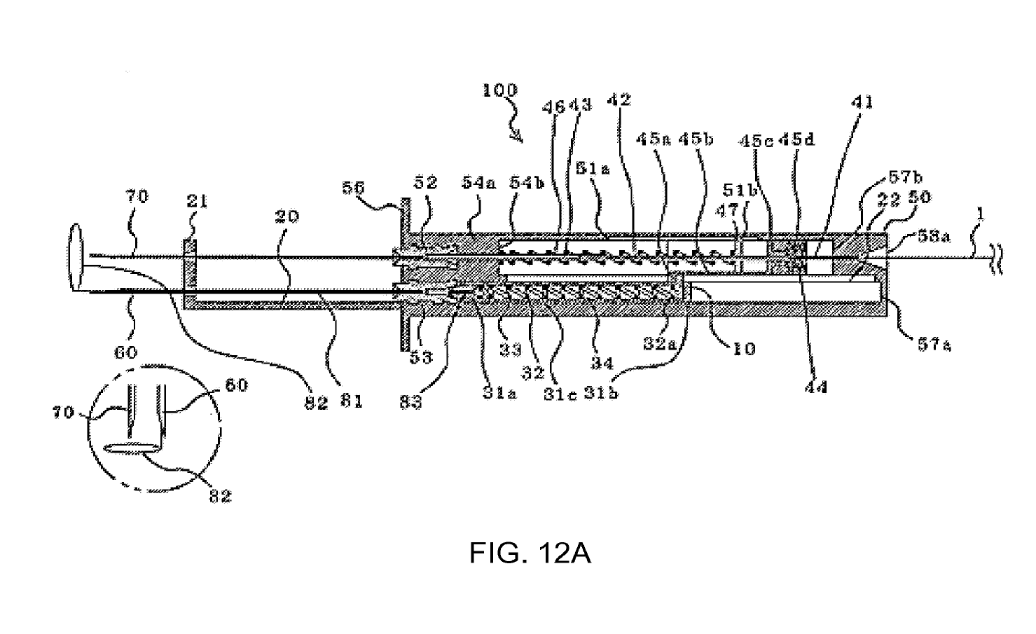

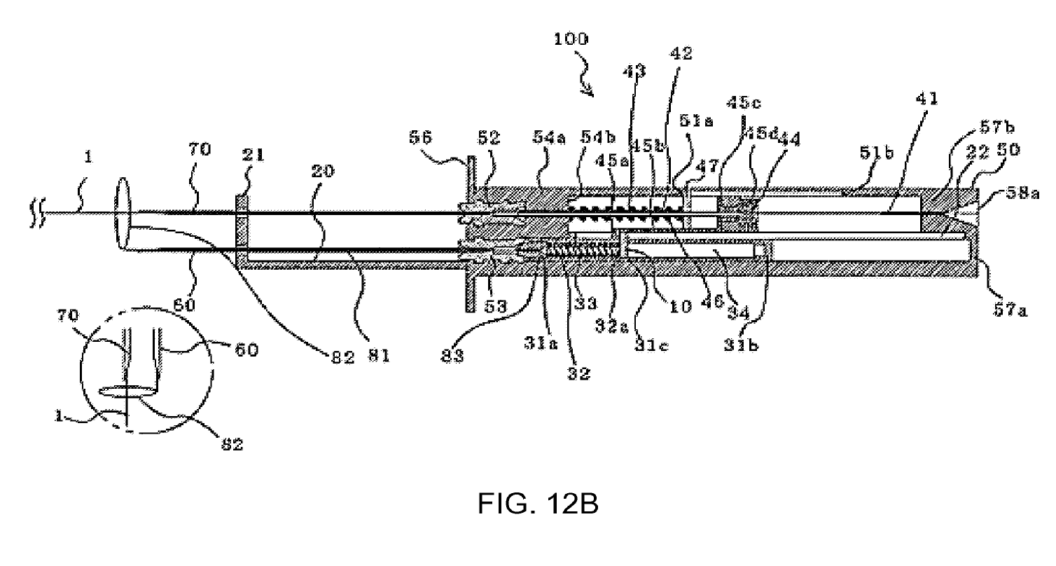

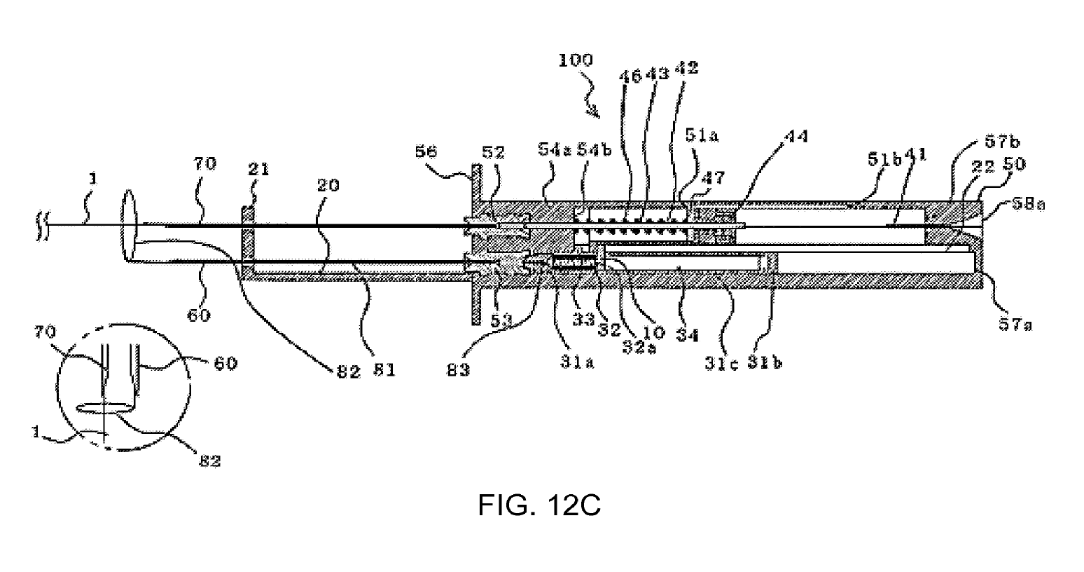

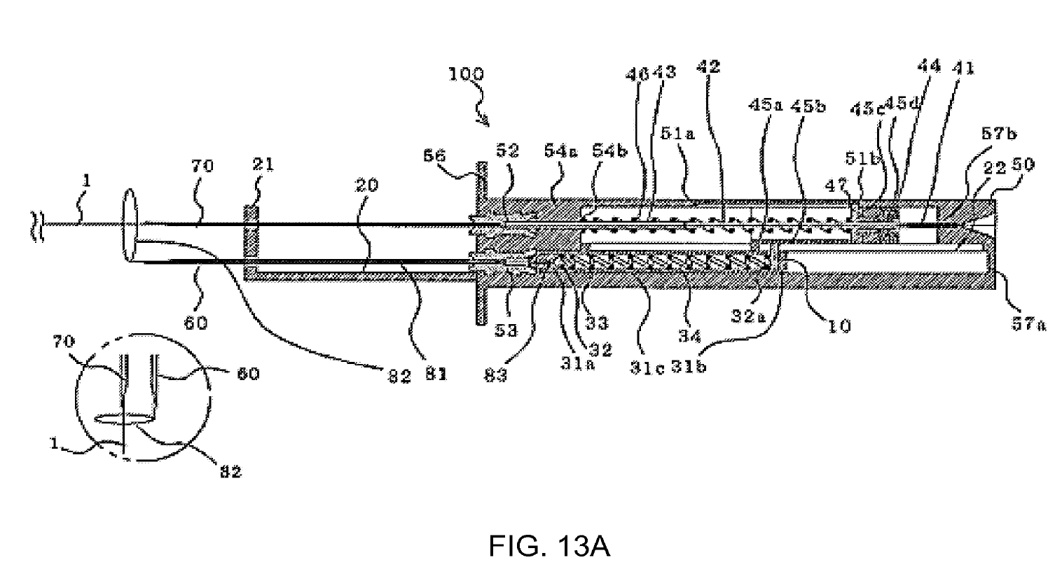

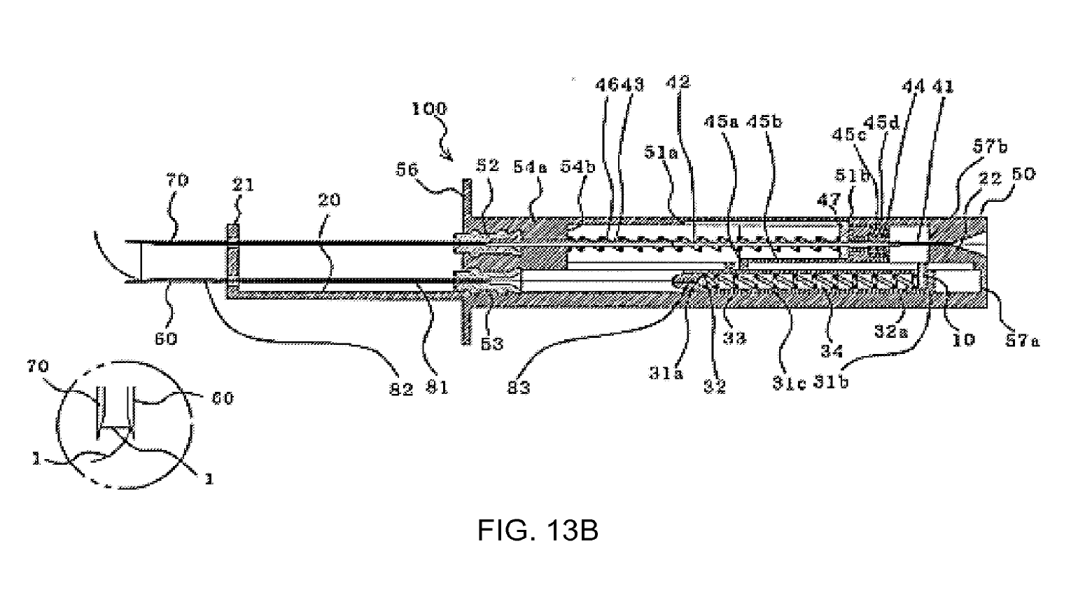

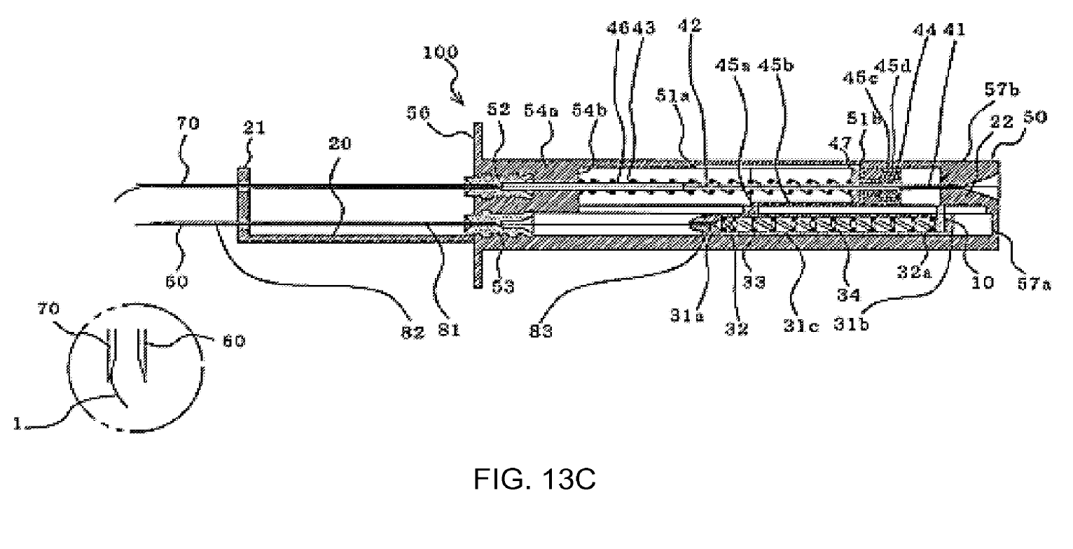

FIGS. 10 to 13C are schematic views in cross section showing, in simplified form, the operating procedure for the suturing instrument 100. The specific operating procedure for the suturing instrument 100 will be described with reference to FIGS. 10 to 13C. FIG. 10 shows the piercing operation of the suturing instrument 100; FIGS. 11A-11C and 12A-12C show the forwards movement operations of each component (referred to below as “pushing operations” (pushing operation 1 (B1)-pushing operation 6 (B6))); and FIGS. 13A-13C show the backwards movement operations of each component (referred to below as “return operations” (return operation 1 (C1)-return operation 3 (C3))). Furthermore, FIGS. 11A to 13C also show diagrams highlighting the fed out state of the suture thread 1 and the loop 82. It should be noted that in FIGS. 11A to 13C, the reference symbols for the loop feed/return mechanism 30, loop feed/return part 31, suture thread feed mechanism 40, suture thread feed part 45 and loop forming part 80 have been omitted for convenience in order to make the state of movement of each component clearer. Furthermore, FIG. 10 also shows the return operation 4 (C4).

According to the illustrated embodiment, the practitioner first of all pierces the patient's body using the suturing instrument 100 which is in a state such that the loop 82 of the loop forming part 80 is accommodated inside the loop introduction needle 60 and the suture thread 1 is accommodated inside the suture thread introduction needle 70. At this point, the suture thread 1 is inserted inside the suture thread introduction needle 70 in such a way as not to project from the tip end of the suture thread introduction needle 70. Moreover, in some embodiments, at least part of the suture thread 1 from the thread dispensing opening 93 to the thread insertion opening 58a is exposed, so the practitioner can adjust the position of the suture thread 1 by gripping the exposed portion of the suture thread 1 and pushing or pulling the suture thread 1.

Furthermore, the suture thread clamping member 44 has over-mounted the first hollow member 41, and the suture thread 1 is free, in other words not moving forwards. In addition, the base end part 31b of the loop feed/return part 31 is in abutment with the inner wall surface 57a.

Referring to FIG. 11A and pushing operation 1 (B1), when a forwards force is applied to the operation part 10 by the practitioner, the loop feed/return part 31 starts to move forwards by way of the first elastic member 32. When the loop feed/return part 31 moves backwards and forwards, the loop forming part 80 also moves backwards and forwards. The loop feed/return part 31 is able to move forwards until the tip end part 31a runs into the support part 53.

The suture thread feed mechanism 40 does not move yet, even though the loop feed/return mechanism 30 has started moving. The suture thread clamping member 44 has over-mounted the first hollow part 41, and at this point the suture thread 1 remains free and no force is transmitted from the operation part 10 to the suture thread 1, so the suture thread 1 does not move.

Referring to FIG. 11B and pushing operation 2 (B2), when the operation part 10 is moved further forwards by the practitioner, the loop forming part 80 which is connected to the tip end part 31a on the loop feed/return mechanism 30 side also moves towards the tip end inside the cavity of the loop introduction needle 60, and is fed out from the tip end of the loop introduction needle 60. When the loop 82 is fed out from the tip end of the loop introduction needle 60, no external force is applied thereto so its annular shape is restored.

In the suture thread feed mechanism 40, the operation part 10 abuts the second abutment part 45a of the suture thread feed mechanism 40 before the loop 82 has been fully restored.

Referring to FIG. 11C and pushing operation 3 (B3), the loop feed/return part 31 continues moving forwards until the first abutment part 33 abuts the inner wall surface 54b.

The suture thread feed mechanism 40 starts to move forwards when the operation part 10 comes into abutment with the second abutment part 45a of the suture thread feed mechanism 40. When the second abutment part 45a is pushed forwards by the operation part 10, the suture thread feed part 45 starts to move forwards. The suture thread clamping member 44 is fitted to the suture thread feed part 45, so the suture thread clamping member 44 starts to move forwards along the first hollow member 41 together with the suture thread feed part 45.

Referring to FIG. 12A and pushing operation 4 (B4), when the operation part 10 is moved further forwards by the practitioner, on the loop feed/return mechanism 30 side, the first abutment part 33 abuts the inner wall surface 54b and the loop feed/return part 31 cannot move any further forwards. When this happens, the movement of the operation part 10 is transmitted only to the first elastic member 32, and the first elastic member 32 is compressed in the forwards direction inside the accommodation part 34 of the loop feed/return part 31. This means that only the first elastic member 32 moves forwards while the shape of the loop 82 is maintained. At this point, the action of the first elastic member 32 causes a load to be gradually exerted by way of the operation part 10 in the practitioner's hand. The operation part 10 can therefore move smoothly forwards.

When the first abutment part 33 abuts the inner wall surface 54b, the forwards movement of the loop feed/return part 31 is regulated, and only the first elastic member 32 moves forwards if the operation part 10 is moved further forwards. That is to say, the loop feed/return part 31 can compress only the first elastic member 32 towards the tip end, without moving any further towards the tip end. As a result, the shape of the loop 82 which has been fed out from the tip end of the loop introduction needle 60 is maintained.

On the suture thread feed mechanism 40 side, the forwards movement of the suture thread feed part 45 continues and the suture thread clamping member 44 separates from the first hollow member 41 to clamp the suture thread 1.

At the same time, the second hollow member 42 also continues to move forwards, so the second elastic member 46 starts to be compressed as the movement regulating member 47 moves forwards.

Referring to FIG. 12B and pushing operation 5 (B5), in the loop feed/return mechanism 30, the movement of the operation part 10 is transmitted only to the first elastic member 32, and the first elastic member 32 continues to be compressed.

In the suture thread feed mechanism 40, if the suture thread clamping member 44 moves further forwards while the suture thread 1 is being clamped by the suture thread clamping member 44, the suture thread 1 which is being clamped by the suture thread clamping member 44 also starts to move forwards, and the suture thread 1 wound on the bobbin 94 inside the thread housing part 90 is fed out from the thread housing part 90. The suture thread 1 is then exposed as it is gradually fed out from the tip end of the suture thread introduction needle 70. The suture thread 1 which has been fed out from the tip end of the suture thread introduction needle 70 passes through the loop 82 which has already been fed out. Moreover, the second hollow member 42 is also pushed forwards by the suture thread clamping member 44 and moves forwards.

In some embodiments, the thread housing part 90 is provided in order to accommodate the suture thread 1 which is fed out from the suture thread introduction needle 70, so the suture thread 1 does not obstruct the practitioner's work and the ease of use is improved. Furthermore, the thread housing part 90 has a circular shape which allows the suture thread 1 to be wound and accommodated in a circular shape, so it is possible to restrict the tendency of the suture thread 1 to retain its wound shape by adjusting the winding diameter of the suture thread 1. By thus restricting the tendency of the suture thread 1 to retain its wound shape, the suture thread 1 which has been fed out from the tip end of the suture thread introduction needle 70 can pass more reliably through the loop 82. Furthermore, the bobbin 94 on which the suture thread 1 is wound is provided, so the suture thread 1 is less likely to become tangled inside the thread housing part 90, and the suture thread 1 can be smoothly fed out.

In accordance with further embodiments, the thread dispensing opening 93 and the thread insertion opening 58a are disposed in such a way that the tangent to the thread housing part 90 at the thread dispensing opening 93 and an imaginary line joining the thread dispensing opening 93 and the thread insertion opening 58a run in substantially the same direction. Consequently, the suture thread 1 which has been wound and accommodated in an annular shape inside the thread housing part 90 is guided to the thread insertion opening 58a with few bends or curves, and the suture thread 1 can be smoothly introduced into the suturing instrument 100.

After this, the suture thread feed part 45 continues moving forwards until the movement regulating member 47 runs into the stopper 51a. At this point, the required length of suture thread 1 has been fed out from the suture thread introduction needle 70.

In accordance with some embodiments, a position of the operation part at this point in the operation of the medical suturing instrument is referred to as a first position. According to these embodiments, with the operation part in the first position, the operation parts has moved the loop feed return mechanism into feeding the loop out from the loop introduction needle, and has also moved the suture thread feed mechanism into feeding the thread out from the suture thread introduction needle.

Referring to FIG. 12C and pushing operation 6 (B6), when the operation part 10 is moved further forwards by the practitioner, the compression of the first elastic member 32 and the forwards movement of the suture thread feed part 45 continue. However, the second hollow member 42 does not move any further forwards because the movement regulating member 47 runs into the stopper 51a. If the suture thread feed part 45 is moved further forwards in this state, the second hollow member 42 penetrates the suture thread clamping member 44. When this happens, the clamping of the suture thread 1 is released and the suture thread 1 is once again free. That is, the suture thread 1 no longer moves forwards. Meanwhile, on the loop feed/return mechanism 30 side, only the first elastic member 32 is compressed while the shape of the loop 82 is maintained. The state of the suture thread 1 having passed through the inside of the loop 82 is therefore maintained.

Referring to FIG. 13A and return operation 1 (C1), when the force towards the tip end applied at the operation part 10 by the practitioner is released, the compressed state of the first elastic member 32 and the second elastic member 46 is lifted. When the compressed state of the first elastic member 32 is lifted, the operation part 10 is pushed by the first elastic member 32 and moves backwards. The operation part 10 then abuts the base end part 31b of the loop feed/return part 31. The second hollow member 42 penetrates the suture thread clamping member 44, and therefore the second hollow member 42 is also moved backwards together with the suture thread clamping member 44. After this, the backwards movement of the suture thread clamping member 44 together with the second hollow member 42, whereof the movement is regulated by the movement regulating member 47 running into the stopper 51b, is stopped for a time.

It should be noted that the figures show an exemplary case in which the suture thread feed part 45 is moved backwards by the lifting of the compressed state of the second elastic member 46, but the suture thread feed part 45 can still move backwards without the second elastic member 46, as will be described later.

Referring to FIG. 13B and return operation 2 (C2), when the operation part 10 is moved further backwards by the practitioner, the operation part 10 pushes the base end part 31b of the loop feed/return part 31, and the loop feed/return part 31 starts to move backwards. As the loop feed/return part 31 moves backwards, the loop 82 starts to be accommodated inside the loop introduction needle 60, in such a way that the suture thread 1 is drawn to the tip end of the loop introduction needle 60.

In accordance with some embodiments, a position of the operation part at a completion of return operation 2 is referred to as a second position. According to these embodiments, with the operation part in the second position, loop feed/return mechanism and the suture thread feed mechanism are retracted to return the loop at least partly inside the loop introduction needle after the suture thread has been gripped by the loop. According to one embodiment, the loop is prevented from being returned fully within the loop introduction needle so long as the suture thread is gripped by the loop. In an alternate embodiment, the loop is fully retracted within the loop introduction needle while gripping the suture thread.

The suturing instrument 100 makes it possible to adjust the timing such that the base end surface of the first abutment part 33 and the tip end surface of the second abutment part 45a come into abutment after the suture thread 1 has been gripped at the tip end of the loop introduction needle 60, or at the same time as the suture thread 1 is gripped at the tip end of the loop introduction needle 60. Consequently, before the suture thread 1 is gripped at the tip end of the loop introduction needle 60, the base end surface of the first abutment part 33 and the tip end surface of the second abutment part 45a are not yet in abutment. That is to say, the backwards force of the operation part 10 is not transmitted to the suture thread feed part 45 before the suture thread 1 has been gripped at the tip end of the loop introduction needle 60.

The base end surface of the first abutment part 33 and the tip end surface of the second abutment part 45a come into abutment after the suture thread 1 has been gripped at the tip end of the loop introduction needle 60, or at the same time as the suture thread 1 is gripped at the tip end of the loop introduction needle 60 (see return operation 3 below). That is to say, in the suturing instrument 100, the feeling when the suture thread 1 is gripped at the tip end of the loop introduction needle 60 is transmitted to the practitioner by way of the operation part 10 before the feeling when the base end surface of the first abutment part 33 and the tip end surface of the second abutment part 45a come into abutment is transmitted to the practitioner by way of the operation part 10, or at the same time as the feeling when the base end surface of the first abutment part 33 and the tip end surface of the second abutment part 45a come into abutment is transmitted to the practitioner by way of the operation part 10.

According to one embodiment, the first abutment part 33 is included as a projection included in the loop feed/return mechanism 30 while the second abutment part 45a is included as a projection included in the suture feed thread mechanism 40. In various embodiments, either or both of the first abutment 33 and the second abutment 45a are configured to extend radially from the loop feed/return mechanism 30 and the suture feed thread mechanism 40, respectively. According to some embodiments, the abutments 33, 45a are in the form of tabs or other similar structure.

When the suture thread 1 has been gripped at the tip end of the loop introduction needle 60, the loop forming part 80 cannot enter the loop introduction needle 60, so neither the loop feed/return part 31 nor the suture thread feed part 45 can move any further backwards, even if the practitioner attempts to move the operation part 10 backwards.

In this state, when the suturing instrument 100 is removed, the suture thread 1 is smoothly guided because it is free, and the suture thread 1 reaches a state of having passed through the internal organ wall and the abdominal wall in substantially a U-shape. That is, the practitioner can make the suture thread 1 reach a state of having passed through the internal organ wall and the abdominal wall in substantially a U-shape simply by means of pushing operations (pushing operations 1-6) and return operations (return operations 1 and 2).

Referring to FIG. 13C and return operation 3 (C3), the practitioner then uses the cutting tip of the suture thread introduction needle 70 which has been withdrawn to outside the body or another cutting means (scissors, cutter etc.) to cut the suture thread 1. The excess suture thread 1 gripped at the tip end of the loop introduction needle 60 is then removed. When this is done, the loop forming part 80 can enter the loop introduction needle 60 and the loop feed/return part 31 can move backwards.

In this state, when the operation part 10 is moved further backwards, the loop feed/return part 31 moves backwards and the first abutment part 33 and second abutment part 45a come into abutment. The backwards force of the operation part 10 is transmitted to the second abutment part 45a by way of the first abutment part 33, and the suture thread feed part 45 moves backwards.

Referring again to FIG. 10 a return operation 4 is illustrated in which each component returns to an initial state. When the suture thread feed part 45 moves further backwards, the suture thread clamping member 44 is withdrawn from the second hollow member 42 and then over-mounts the first hollow member 41, in the same way as in the initial state. The suture thread 1 remains free. That is, the suture thread 1 does not move.

Meanwhile, the loop feed/return part 31 moves backwards, under the force of the first elastic member 32 and the backwards force applied to the operation part 10, until the backwards movement of the suture thread feed part 45 stops. When the suture thread clamping member 44 returns to the initial state, the parts return to the initial state together (see FIG. 10).

Accordingly, the same state as the initial state is reached, in which the suture thread 1 is inserted up to the tip end of the suture thread introduction needle 70, and therefore the internal organ wall can be fixed any number of times using the above procedure. Moreover, if the suture thread 1 is projecting further towards the tip end than the cutting edge of the suture thread introduction needle 70 after the suture thread 1 has been cut, the same state as the initial state may be set by pulling the suture thread 1 towards the base end by hand.

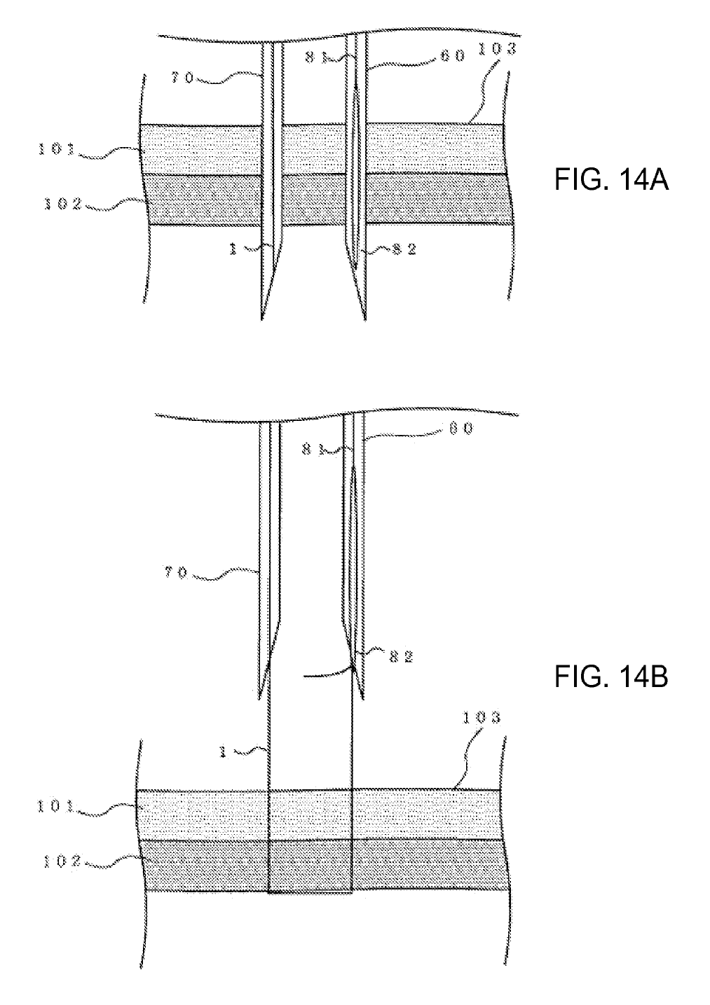

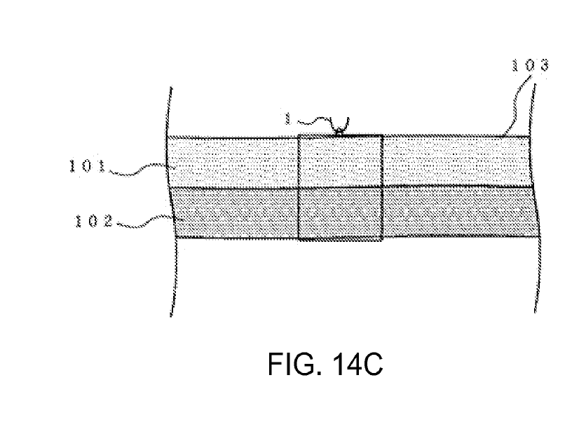

FIGS. 14A-14C are explanatory diagrams to illustrate the action of the suturing instrument 100 according to one embodiment. The action of the suturing instrument 100 will be described in line with the flow of the procedure employing the suturing instrument 100, with reference to FIGS. 14A-14C. In the description given here, a patient's abdominal wall 101 and internal organ wall 102 (e.g., stomach wall) are fixed using the suturing instrument 100.

The practitioner first of all inserts an endoscope orally or nasally into the patient's internal organ (e.g., the stomach). The practitioner then expands the internal organ by filling the internal organ with a gas (e.g., carbon dioxide). As a result, the internal organ wall 102 is placed in close contact with the abdominal wall 101. Next, the skin 103 is sterilized, including the site to be pierced by the loop introduction needle 60 and the suture thread introduction needle 70. The position of the internal organ is then confirmed by the light emitted from the endoscope, and a local anaesthetic is administered at this site.

Next, the suturing instrument 100 is prepared: the loop 82 of the loop forming part 80 is accommodated inside the loop introduction needle 60, and the suture thread 1 is accommodated inside the suture thread introduction needle 70 (the state shown in FIG. 10). At this point, the suture thread 1 is accommodated inside the suture thread introduction needle 70 in such a way as not to project from the tip end of the suture thread introduction needle 70. The loop introduction needle 60 and the suture thread introduction needle 70 of the suturing instrument 100 are then made to pierce the patient's abdominal wall 101, and the loop introduction needle 60 and the suture thread introduction needle 70 are made to project into the internal organ from the internal organ wall 102 (FIG. 14A).

Once this state has been confirmed using the endoscope, the practitioner moves the operation part 10 forwards. The practitioner then carries out the series of operations shown in FIGS. 11A to 13C, and the suture thread 1 is exposed outside the body (FIG. 14B). That is, the suture thread 1 is in the situation of passing through the internal organ wall 102 and the abdominal wall 101 in substantially a U-shape. Finally, the practitioner cuts the suture thread 1 which has been withdrawn to outside the body and ties the two ends of the suture thread 1 (FIG. 14C). The internal organ wall 102 and the abdominal wall 101 are fixed by this tying.

The suturing instrument 100 is then once again made to pierce the body substantially parallel to the position of the tie at a prescribed distance of around 20-30 mm, for example, and the internal organ wall 102 and the abdominal wall 101 are fixed. That is, the suture thread 1 functions as an organopexy tool in order to fix the internal organ wall 102 and the abdominal wall 101, so it is possible to easily form a fistula which is used when inserting a fistula catheter. In this process, by placing a length of suture thread 1 inside the thread housing part 90 which provides for multiple fixing operations, it is possible to use the suturing instrument 100 continuously without having to insert the suture thread 1 into the suturing instrument 100 each time a fixing operation is carried out. Furthermore, a long piece of suture thread 1 is placed inside the thread housing part 90, so the practitioner's productivity can be satisfactorily maintained without any obstruction to the practitioner's work.

The suturing instrument 100 makes it possible to fix internal organs any number of times until the suture thread 1 runs out, without the need to insert the suture thread 1 into the suturing instrument 100 every time. Furthermore, the suturing instrument 100 makes it possible to pass the suture thread 1 in a U-shape through the internal organ wall 102 and the abdominal wall 101 simply by means of operations involving pushing and pulling of the operation part 10 (pushing operations (pushing operations 1-6) and return operations (return operations 1, 2)). Specifically, the practitioner can pass the suture thread 1 through the internal organ wall 102 and the abdominal wall 101 simply by carrying out an operation to move the operation part 10 forwards and an operation until a feeling of the suture thread 1 being gripped at the tip end of the loop introduction needle 60 is transmitted, so the procedure which the practitioner needs to follow can be considerably simplified. In addition, the suture thread 1 is fed out once the loop 82 has already opened into an annular shape, so the suture thread 1 can be reliably inserted into the loop 82.

Accordingly, the burden on the practitioner can be considerably reduced and the ease of use is markedly improved. Furthermore, the procedure itself can be carried out very simply, and therefore not only is the ease of use improved, but the ease of handling is also considerably improved. In addition to the procedure being simple, the suture thread 1 can also be securely gripped, and therefore it is possible to minimize the number of times piercings are made, so this can contribute not only to reducing the burden on the practitioner, but also to reducing the burden on the patient. In addition, the first elastic member 32 is provided, so the action of the first elastic member 32 enables the pushing operations to be smoothly carried out and improves stability during the procedure.

With the suturing instrument 100, by placing a length of suture thread 1 inside the thread housing part 90 which provides for multiple fixing operations, it is possible to use the suturing instrument 100 continuously without having to insert the suture thread 1 into the suturing instrument 100 each time a fixing operation is carried out. Furthermore, a long piece of suture thread 1 providing for multiple fixing operations is placed inside the thread housing part 90, so the practitioner's productivity can be satisfactorily maintained without a long piece of the suture thread 1 obstructing the practitioner's work.

In accordance with the embodiments described above, the bobbin 94 on which the suture thread 1 is wound is provided. However, in accordance with an alternate embodiment, the bobbin 94 is not provided. It should be noted that the description concerning embodiments in which a bobbin is not provided focuses on the differences between these embodiments and those above that describe embodiments including a bobbin. Furthermore, constituent parts which are the same as in the various embodiments bear the same reference symbols.

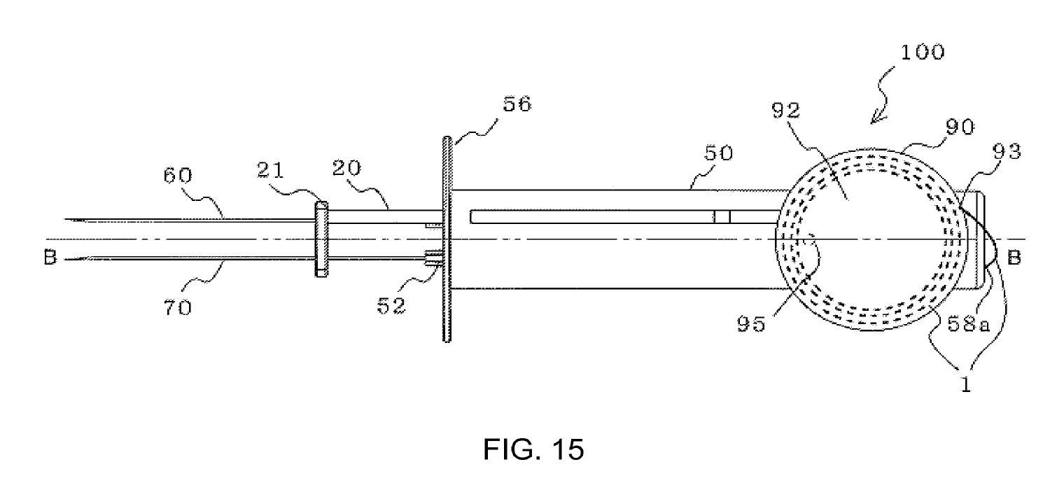

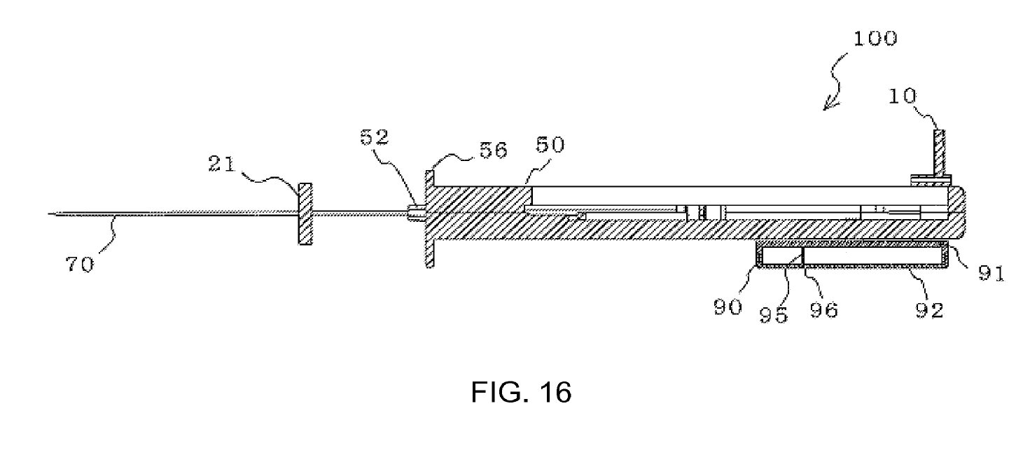

FIG. 15 illustrates a bottom view of the suturing instrument 100 according to embodiments in which a bobbin is not provided. FIG. 16 is a view in cross section along B-B in FIG. 15. It should be noted that the suture thread 1 is shown by the broken line in FIG. 15.

A pin 95 standing upright in a substantially vertical direction is provided inside the thread housing part 90. As shown in FIG. 15, the suture thread 1 which is wound in a substantially annular state is disposed further towards the outer circumferential side inside the substantially circular thread housing part 90 than the pin 95. According to these embodiments, the pin 95 is formed as a single piece with the housing part 91 in such a way as to stand upright from the bottom surface of the housing part 91. Furthermore, in accordance with this embodiment, the cover 92 is provided with a fitting hole 96 in which the tip end of the pin 95 is fitted. In this way, there is no gap above or below the pin 95 inside the thread housing part 90 and the suture thread 1 which is provided wound in an annular shape around the outer periphery of the pin 95 does not become detached from the pin 95.

Moreover, in some embodiments, the pin 95 does not have to be provided on the bottom surface of the housing part 91 in the thread housing part 90, and alternatively, may be provided on the inner surface of the cover 92. Furthermore, if the cover 92 is not detachable from the housing part 91, the two ends of the pin 95 may be joined to the housing part 91 and cover 92.

By providing this kind of pin 95, it is possible to restrict tangling of the suture thread 1 which is wound in an annular shape when the suture thread 1 is pulled out from the thread dispensing opening 93 in the thread housing part 90. Because these embodiments do not include the bobbin 94, it is possible to curb increases in the number of components in the suturing instrument 100.

It should be noted that the positioning of the pin 95 in the thread housing part 90 should be determined taking account of the positional relationship with the thread dispensing opening 93. Specifically, the pin 95 should be positioned in such a way that the orientation of the suture thread 1 from the pin 95 to the thread dispensing opening 93 is close to the orientation of the suture thread 1 from the thread dispensing opening 93 to the thread insertion opening 58a. The suture thread 1 is smoothly guided to the thread insertion opening 58a as a result. Furthermore, multiple pins 95 may be provided. By providing multiple pins 95, the suture thread 1 which has been wound in an annular shape is easily maintained in that shape and it is possible to enhance the effect of restricting tangling of the suture thread 1.

According to the embodiments described above, operation of the suturing instrument 100 automatically dispenses suture thread 1 stored in the thread housing part 90. Such an approach frees the practitioner or an assistant from the labor and attention required to feed suture thread 1 into the suturing instrument 100 when the instrument is in use. Further, in some embodiments, a single operating stroke (for example, operation of the operating part 10 as illustrated in FIGS. 11A-13C and pushing operation 1 (B1)-pushing operation 6 (B6) followed by return operation 1 (C1)-return operation 3 (C3)) results in an automatic feed of the suture thread and capture of the suture thread by the loop. The preceding makes a stitching operation on a body and associated internal organs more failsafe and faster to complete.

### DESCRIPTION OF REFERENCE NUMERALS

1 suture thread, 10 operation part, 20 support rod, 21 flat plate part, 22 groove, 30 loop feed/return mechanism, 31 loop feed/return part, 31a tip end part, 31b base end part, 31c side wall part, 32 first elastic member, 32a base end part, 33 first abutment part, 34 accommodation part, 40 suture thread feed mechanism, 41 first hollow member, 42 second hollow member, 43 third hollow member, 44 suture thread clamping member, 45 suture thread feed part, 45a second abutment part, 45b side wall extension part, 45c main body part, 45d fitting part, 46 second elastic member, 47 movement regulating member, 50 housing, 51a stopper, 51b stopper, 52 support part, 53 support part, 54a thick-walled part, 54b tip end inner wall surface, 55 guide hole, 56 flange part, 57a base end inner wall surface, 57b base end inner wall surface, 58 through-hole, 58a thread insertion opening, 60 loop introduction needle, 70 suture thread introduction needle, 80 loop forming part, 81 rod-like shaft, 82 loop, 83 base end fixing part, 90 thread housing part, 91 housing part, 92 cover, 93 thread dispensing opening, 94 bobbin, 94a thread winding part, 94b flange part, 94c notch, 95 pin, 96 fitting hole, 100 suturing instrument, 101 abdominal wall, 102 internal organ wall, 103 skin.

1. A medical suturing instrument comprising:
a loop introduction needle;
a suture thread introduction needle configured to receive suture thread inserted therewithin;
a housing configured to hold the loop introduction needle and the suture thread introduction needle substantially parallel a prescribed distance apart;
a loop forming part having a loop formed at the tip end thereof, the loop forming part movably housed inside the loop introduction needle;
a loop feed/return mechanism located inside the housing and configured to move the loop forming part in an axial direction;
a suture thread feed mechanism located inside the housing and configured to feed out the suture thread on a tip end side; and
a thread housing part coupled to the housing for accommodating the suture thread which is supplied to the suture thread feed mechanism.

2. The medical suturing instrument according to claim 1, further comprising an operation part configured to receiving input operations,
wherein the operation part is configured to move in a forward direction on receipt of an input operation to move the loop feed/return mechanism into feeding the loop out from the loop introduction needle, and to move the suture thread feed mechanism into feeding the suture thread out from the suture thread introduction needle after at least part of the loop has projected from a tip end of the loop introduction needle,
wherein the suture thread feed mechanism is configured to operate to enable the suture thread to freely advance and retract after the loop is fed out from the loop introduction needle and the suture thread is fed out from the suture thread introduction needle, and
wherein the loop feed/return mechanism is configured to operate to return the loop inside the loop introduction needle after the loop is fed out from the loop introduction needle and the suture thread is fed out from the suture thread introduction needle.

3. The medical suturing instrument according to claim 2, further comprising:
a bobbin located inside the thread housing and configured to have the suture thread wound thereon and to rotate in a winding direction of the suture thread; and
a pin located inside the thread housing part and extending in a direction which intersects a winding direction of the suture thread when housed in a wound state inside the thread housing part.

4. The medical suturing instrument according to claim 1, wherein the thread housing part comprises:
a peripheral wall which has a substantially circular planar shape;
a thread dispensing opening configured to dispense the suture thread inside the thread housing part and located in the peripheral wall of the thread housing part; and
a thread insertion opening located in the housing and configured to receive suture thread inserted into the housing, and
wherein the thread dispensing opening and the thread insertion opening are located in such a way that when viewed as a plane, an imaginary line joining the two substantially coincides with the tangential direction of the peripheral wall at the thread dispensing opening.

5. The medical suturing instrument according to claim 4, further comprising a bobbin located inside the thread housing and configured to have the suture thread wound thereon and to rotate in a winding direction of the suture thread.

6. A method for thread-feeding and thread-storage in a medical suturing instrument including a loop introduction needle, a suture thread introduction needle, a housing configured to hold the loop introduction needle and the suture thread introduction needle, a loop forming part having a loop at a tip end thereof, a loop feed/return mechanism inside the housing, a suture thread feed mechanism inside the housing and configured to feed out the suture thread on a tip end side and a thread housing part located in the housing, the method comprising:
coupling the loop introduction needle and the suture thread introduction needle to the housing substantially parallel to one another a prescribed distance apart;
movably housing the loop forming part within the loop introduction needle;
housing the suture thread in the thread housing part;
operating the medical suturing instrument to move the loop forming part in an axial direction with the loop feed/return mechanism; and
operating the medical suturing instrument to withdraw suture thread from the thread housing part and feed the suture thread into the suture thread introduction needle using the suture thread feed mechanism.

7. The method of claim 6, wherein the medical suturing instrument further comprises an operating part and a bobbin located within the thread housing part, and wherein the method further comprises:
rotating the bobbin in a winding direction of the suture thread to wind the suture thread on the bobbin;
moving the operating part in a forward direction on receipt of an input operation;
moving the loop feed/return mechanism into feeding the loop out from the loop introduction needle;
moving the suture thread feed mechanism into feeding the suture thread out from the suture thread introduction needle after at least part of the loop has projected from a tip end of the loop introduction needle;
operating the suture thread feed mechanism to enable the suture thread to freely advance and retract after the loop is fed out from the loop introduction needle and the suture thread is fed out from the suture thread introduction needle; and
operating the loop feed/return mechanism to return the loop inside the loop introduction needle after the loop is fed out from the loop introduction needle and the suture thread is fed out from the suture thread introduction needle.

8. The method of claim 7, further comprising locating a pin inside the thread housing part extending in a direction which intersects a winding direction of the suture thread when housed in a wound state inside the thread housing part.

9. The method of claim 6, further comprising:
including a thread insertion opening in the housing;
including a peripheral wall having a substantially circular planar shape and a thread dispensing opening located therein within the thread housing part;
locating the thread dispensing opening and the thread insertion opening relative to one another such that, when viewed as a plane, an imaginary line joining the two substantially coincides with the tangential direction of the peripheral wall at the thread dispensing opening; and
dispensing the suture thread via the thread dispensing opening and inserting the suture thread into the housing via the thread insertion opening.

10. A medical suturing instrument characterized in that it comprises:
a loop introduction needle;
a suture thread introduction needle into which suture thread is inserted;
a housing for holding the loop introduction needle and the suture thread introduction needle substantially parallel a prescribed distance apart;
a loop forming part which has a loop formed at the tip end thereof and is movably housed inside the loop introduction needle;
a loop feed/return mechanism provided inside the housing for moving the loop forming part in the axial direction;
a suture thread feed mechanism provided inside the housing for feeding out the suture thread on the tip end side; and
a thread housing part provided in the housing for accommodating the suture thread which is supplied to the suture thread feed mechanism.

11. The medical suturing instrument according to claim 10, characterized in that it comprises an operation part for receiving input operations, and when the operation part receives an input operation, the loop feed/return mechanism operates in such a way that the loop projects from the tip end of the loop introduction needle, and after at least part of the loop has projected from the tip end of the loop introduction needle, the suture thread feed mechanism also operates in such a way that the suture thread projects from the tip end of the suture thread introduction needle;
after this, the suture thread feed mechanism operates in such a way as to enable the suture thread to freely advance and retract; and
after this, the loop feed/return mechanism operates in such a way that the loop returns inside the loop introduction needle.

12. The medical suturing instrument according to claim 10, characterized in that a bobbin on which the suture thread is wound is provided inside the thread housing part in such a way as to be able to rotate in the winding direction of the suture thread.

13. The medical suturing instrument according to claim 10, characterized in that a pin extending in a direction which intersects the winding direction of the suture thread housed inside the thread housing part in a wound state is provided inside the thread housing part.

14. The medical suturing instrument according to claim 10, wherein the thread housing part includes a peripheral wall which has a substantially circular planar shape;
thread dispensing opening for dispensing the suture thread inside the thread housing part is provided in the peripheral wall of the thread housing part;
thread insertion opening for inserting the suture thread into the housing is provided in said housing; and
the thread dispensing opening and thread insertion opening are provided in such a way that when viewed as a plane, an imaginary line joining the two substantially coincides with the tangential direction of the peripheral wall at the thread dispensing opening.

15. A method of making a medical suturing instrument, the method comprising:
providing each of a loop introduction needle and a suture thread introduction needle, the suture thread needle configured to receive suture thread inserted therewithin;
housing the loop introduction needle and the suture thread introduction needle, at least partly, in a housing such that loop introduction needle and the suture thread introduction needle are held substantially parallel to one another a prescribed distance apart;
locating a loop forming part inside the loop introduction needle, the loop forming part having a loop formed at a tip end thereof, the loop forming part movably housed inside the loop introduction needle;
locating each of a loop feed/return mechanism and a suture thread feed mechanism inside the housing, the loop fed return mechanism configured to move the loop forming part in an axial direction, the suture thread feed mechanism configured to feed out the suture thread on a tip end side; and
coupling a thread housing part to the housing, the thread housing part configured to accommodate a suture thread which is supplied to the suture thread feed mechanism.
